(19) United States
(12) Patent Application Publication
(10) Pub. No.: US 2026/0366029 A1
(43) Pub. Date: Nov. 19, 2026

(54) EPH2A APTAMER AND USES THEREOF

(71) Applicants: FUNDACIÓ INSTITUT D'INVESTIGACIÓ BIOMÈDICA DE BELLVITGE (IDIBELL), L'Hospitalet de Llobregat (ES); FUNDACION ALBA PEREZ, LUCHA CONTRA EL CANCER INFANTIL, L'Hospitalet de Llobregat (ES); UNIVERSITY OF IOWA RESEARCH FOUNDATION, Iowa City, IA (US)

(72) Inventors: Óscar MARTINEZ TIRADO, Esplugues de Llobregat (ES); Paloma H. GIANGRANDE, Belmont, MA (US)

(21) Appl. No.: 19/646,314

(22) Filed: Apr. 13, 2026

Related U.S. Application Data
(62) Division of application No. 17/616,599, filed on Jun. 2, 2020, now abandoned.

(30) Foreign Application Priority Data
Jun. 3, 2019 (EP) 19382451.3

(51) Int. Cl. C12N 15/115; A61P 35/00; A61P 35/04; C12N 15/113; G01N 33/575

(52) U.S. Cl. CPC C12N 15/115; A61P 35/00; A61P 35/04; C12N 15/1135; G01N 33/57557; C12N 2310/14; C12N 2310/16; C12N 2310/334; C12N 2310/335; C12N 2310/3519

(57) ABSTRACT

The present invention belongs to the field of genetic therapy. In particular, the invention refers to EphA2 specific RNA-based constructs, which are useful for the treatment, prevention and diagnosis of EphA2 expressing cancers.

### CROSS-REFERENCE TO RELATED APPLICATIONS

The present application is a divisional of U.S. patent application Ser. No. 17/616,599 filed Dec. 3, 2021, which is a 35 U.S.C. § 371 national-phase filing of International Application No. PCT/EP2020/065132, filed on Jun. 2, 2020, which claims the benefit of European Patent Application EP19382451.3, filed on Jun. 3, 2019, all of which are incorporated herein by reference in their entirety.

### INCORPORATION BY REFERENCE

The application contains a Sequence Listing which has been submitted electronically in .XML format and is hereby incorporated by reference in its entirety. Said .XML copy, created on Apr. 13, 2026, is named “108663.00376.xml” and is 99,903 bytes in size. The sequence listing contained in this .XML file is part of the specification and is hereby incorporated by reference herein in its entirety.

### FIELD OF THE INVENTION

The present invention belongs to the field of genetic constructs and therapy. In particular, it refers to an RNA-aptamer which specifically binds to EphA2, and uses thereof.

### BACKGROUND OF THE INVENTION

Ephrin (Eph) receptors are the most extensive subfamily of receptor tyrosine-kinases involved in several processes, including angiogenesis, tissue-border formation, cell migration and cell plasticity. These receptors are well-established mediators in cell-cell interactions and motility and are expressed in human cancers, such as melanoma, prostate, breast, colon, lung and esophageal carcinomas. Among these receptors, EphA2 (ephrin type-A receptor 2) has been implicated in many processes crucial to malignant progression, such as migration, invasion, metastasis, proliferation, survival, and angiogenesis. To this end, inhibition of EphA2 leads to decreased tumor growth, survival, and tumor-induced angiogenesis in multiple preclinical models of breast, ovarian, and pancreatic cancers (Tandon et al.; Kasinski and Slack; Quinn et al.). Higher treatment doses are often administered to patients with high-grade disease; these patients often suffer from toxicity due to non-specific targeting to normal tissues. This highlights the need for developing new modalities with improved safety and efficacy profiles.

Sarcomas are rare high-grade tumors, which have a high rate of morbidity and mortality. Their overall incidence has been increasing at an estimated rate of 26% over the last 2 decades. One third of sarcomas falls in a category of low mutation burden and is characterized by specific recurrent genetic changes known as chromosomal translocations. The sarcomas of this category are known as translocation-associated sarcomas (TAS hereinafter), which includes, amongst others, Ewing sarcoma (ES hereinafter), alveolar rhabdomyosarcoma (ARMS hereinafter), synovial sarcoma (SS hereinafter). Two very important properties of these chromosomal translocations (and their associated fusion products) are their consistency and specificity. Multiple studies have indicated that the same translocation (or in some cases, one of a related group of translocations) occurs in most of cases of a given sarcoma, and thus a translocation or group of translocations is consistent within a sarcoma category (Xiao et al., the exact position in the chromosome and the resulting fusion of these translocations is disclosed in this reference and are incorporated herein by reference). Furthermore, this translocation or one of a related group of translocations does not occur in any other type of sarcoma, and thus the translocation is specific for the sarcoma category. Therefore, there is a very close relationship between the translocation or its fusion product and the sarcoma category.

Recently, it has been shown that EphA2 is expressed in ES cells and is essential for the aggressive properties of ES in a kinase-independent manner. Therefore, blocking EphA2 expression or its functions may be of therapeutic use for the treatment of ES (Garcia-Monclús et al.).

Recent advances in RNA technologies offer new and promising tools for developing therapies against sarcomas and other cancers. One of these technologies is aptamer technology. As therapeutic reagents, RNA aptamers have several advantages over small molecule inhibitors or protein-based reagents. Unlike most small molecule inhibitors, aptamers are highly specific and can be used for targeted therapy. In contrast to antibodies, aptamers can be readily chemically synthesized and are amenable to chemical modifications that make them resistant to nucleases and improve their pharmacokinetics in vivo. In addition, chemically modified RNA aptamers have little-to-no immunogenicity and are thus much safer for clinical applications.

However, even though aptamers are known to be powerful therapeutic tools with, at least, the advantages indicated above, there is still pending in the state of the art an effective cancer treatment using RNA-aptamers.

### SUMMARY OF THE INVENTION

Interestingly, the authors of the present invention have developed RNA-aptamers and constructs based on them which are useful in the treatment, prevention and diagnosis of cancer, in particular EphA2 expressing cancer.

Up to now, all the attempts had been focused on using the aptamers as EphA2-targeting carrier to EphA2-expressing cells.

Surprisingly, the inventors have found that aptamers comprising the sequence SEQ ID NO: 1 are able to exert, by their own, a remarkable therapeutic effect on EphA2-expressing cancer cells. Example 4, FIG. 3C, shows that the administration of an aptamer comprising the sequence SEQ ID NO: 1 reduces the clonogenic ability of the tumor cells. This reduction in the clonogenic activity of cancer cells was confirmed incorporating the SEQ ID NO: 1 within a complex comprising, in addition to the aptamer, a siRNA. As it can be concluded from FIG. 10, the clonogenic activity of the EphA2-expressing cancer cells was dramatically reduced when the complex included the sequence SEQ ID NO: 1.

Example 6 below shows that the administration of an aptamer comprising the sequence SEQ ID NO: 1 delays the development of tumors.

It is the first time that it is reported a RNA-aptamer with such therapeutic behavior on the basis of its binding to EphA2-expressing cancer cells.

Thus, in a first aspect the present invention refers to a RNA-aptamer which specifically binds to EphA2, which:
(i) consists of sequence SEQ ID NO: 1; or, alternatively,
(ii) consists of sequence SEQ ID NO: 1 and the pyrimidine moiety of at least one of the nucleotides forming the sequence is a substituted pyrimidine; or, alternatively,
(iii) comprises the sequence SEQ ID NO: 1, and the pyrimidine moiety of at least one of the nucleotides forming the sequence is a substituted pyrimidine;
   wherein the term “substituted pyrimidine” is a pyrimidine of formula (I) when the nucleotide is a cytosine, or of formula (II) when the nucleotide is an uracil

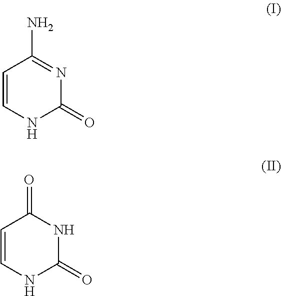 [SMILES: Nc1cc[nH]c(=O)n1 ; O=c1cc[nH]c(=O)[nH]1]

where at least one of the hydrogen radicals bound to at least one of the carbon or nitrogen atoms forming the pyrimidine ring of formula (I) or (II) is substituted by a radical other than hydrogen which confers to the aptamer stability against degradation. Any of the radicals which have already been reported in the prior art as improving aptamer stability (in vitro or in vivo) by substituting pyrimidine ring can be used as the “radical other than hydrogen”.

The inventors performed a structural analysis and concluded that SEQ ID NO:1, which acquired a loop secondary structure, bound to EphA2 protein. The binding to EphA2 is essential in order to internalize the cell. But the aptamer of the invention not only is able to be internalized, as other targeting elements, but that it is able, once within the EphA2-expressing cell, of providing an anti-cancer effect by its own.

The technical effect conferred by sequence SEQ ID NO: 1, in terms of binding to EphA2 and internalization, is so robust that it is found the same behavior both when it is tested forming part of a longer aptamer (SEQ ID NO: 4) and when it is tested forming part of larger constructs (as can be complex of sequence SEQ ID NO: 17). In both cases it is maintained the ability of efficient binding to EphA2-expressing cancer cells, and internalizing cell.

The invention also provides an RNA-aptamer which binds specifically to EphA2 and which:
(i) consists of sequence SEQ ID NO: 1; or
(ii) comprises sequence SEQ ID NO 2 optionally comprising one, two or three substitutions located within any of the positions 1-20 and 46-51 of sequence SEQ ID NO 2.

In addition to the above, FIG. 10 also shows that the aptamer of the invention not only carries the siRNA to the target cells, but also that both the aptamer and the siRNA can exert the beneficious therapeutic effect on the cancer cell once they have been internalized. FIG. 3B already shows that when the aptamer is internalized there is a substantial reduction of the clonogenic ability of the cancer cells, ability which is almost completely null when both, the aptamer and the siRNA (forming part of the complex), are internalized in the cancer cells (FIG. 10). This is indicative of the therapeutic efficiency of the aptamer alone (FIG. 3B) but also of the aptamer in combination with the functional substance, i.e. siRNA (FIG. 10).

In addition to the above, these data also support that the aptamer of the invention can also act as efficient delivery carrier of functional substances. This is also of great importance because the state of the art has reported several drawbacks related to the stability and safe delivery of anti-cancer therapeutic molecules. For example, siRNAs have been reported as being highly unstable as they can rapidly be degraded once administered. The prior art has taught the use of liposomes to protect them from degradation, but the encapsulation in liposomes has been reported as toxic.

Advantageously, the aptamer of the invention allows the safe and stable delivery of functional substances, thus overcoming the drawbacks of the delivery carriers reported up to now.

Thus, in a second aspect, the present invention refers to a complex comprising the RNA-aptamer of the invention, coupled to a functional substance.

In a third aspect, the present invention refers to a composition comprising the aptamer or the complex of the invention.

In a further aspect the present invention provides a RNA-aptamer, which specifically binds to EphA2 and which comprises or consists of sequence SEQ ID NO: 1, wherein optionally one or more of the nucleotides forming the sequence of the aptamer are modified nucleotides; or a composition comprising said aptamer or complex; for use in therapy or diagnostics. In the present invention the expression “modified nucleotide” refers to a nucleotide which differ from the one located in the same position in sequence SEQ ID NO:1 by a chemical modification in the sugar or base moiety, among others. It is well-established such chemical modifications responsible for the aptamer stabilization.

In a fourth aspect, the present invention refers to a RNA-aptamer which specifically binds to EphA2 and which comprises or consists of sequence SEQ ID NO: 1, wherein optionally one or more of the nucleotides forming the sequence of the aptamer is a modified nucleotide; or a complex comprising said aptamer coupled to a functional substance; or a composition comprising said aptamer or complex, for use in the treatment or prevention of cancer or cancer metastasis, wherein the cancer is characterised by expressing EphA2. This aspect can alternatively be formulated as a method for the treatment or prevention of cancer or cancer metastasis, wherein the cancer is characterized by expressing EphA2, the method comprising the administration to a subject in need thereof of a therapeutically effective amount of a RNA-aptamer which specifically binds to EphA2 and which comprises or consists of sequence SEQ ID NO: 1, wherein optionally one or more of the nucleotides forming the sequence of the aptamer is a modified nucleotide; or a complex comprising said aptamer coupled to a functional substance; or a composition comprising said aptamer or complex; to a subject in need thereof. This aspect can alternatively be formulated also as the use of a RNA-aptamer which specifically binds to EphA2 and which comprises or consists of sequence SEQ ID NO: 1, wherein optionally one or more of the nucleotides forming the sequence of the aptamer is a modified nucleotide; or a complex comprising said aptamer coupled to a functional substance; or a composition comprising said aptamer or complex; in the manufacture of a medicament for the treatment or prevention of cancer or cancer metastasis, wherein the cancer is characterized by expressing EphA2.

In a fifth aspect, the present invention refers to the use of a RNA-aptamer which specifically binds to EphA2 and which comprises or consists of sequence SEQ ID NO: 1, wherein optionally one or more of the nucleotides forming the sequence of the aptamer is a modified nucleotide; or a complex comprising said aptamer coupled to a functional substance; or a composition comprising said aptamer or complex, for in vitro or ex vivo diagnosis of cancer or cancer metastasis, wherein the cancer is characterised by expressing EphA2. This aspect can be alternatively formulated as a method for the in vitro or ex vivo diagnosis of cancer or cancer metastasis in a subject, wherein the cancer is characterized by expressing EphA2, the method comprises contacting an isolated test sample of the subject with a RNA-aptamer which specifically binds to EphA2 and which comprises or consists of sequence SEQ ID NO: 1, wherein optionally one or more of the nucleotides forming the sequence of the aptamer is a modified nucleotide; or a complex comprising said aptamer coupled to a functional substance; or a composition comprising said aptamer or complex; and detecting the location of the aptamer or complex.

In a sixth aspect, the present invention refers to a RNA-aptamer which specifically binds to EphA2 and which comprises or consists of sequence SEQ ID NO: 1, wherein optionally one or more of the nucleotides forming the sequence of the aptamer is a modified nucleotide; or a complex comprising said aptamer coupled to a functional substance; or a composition comprising said aptamer or complex; for use in a method of diagnosis in vivo of a cancer characterised by expressing EphA2. This aspect can alternatively be formulated as a method for the in vivo diagnosis of cancer or cancer metastasis in a subject, wherein the cancer is characterized by expressing EphA2, the method comprising administering a RNA-aptamer which specifically binds to EphA2 and which comprises or consists of sequence SEQ ID NO: 1, wherein optionally one or more of the nucleotides forming the sequence of the aptamer is a modified nucleotide; or a complex comprising said aptamer coupled to a functional substance; or a composition comprising said aptamer or complex; and detecting the location of the aptamer or complex.

In a seventh aspect, the present invention refers to a diagnostic kit comprising a RNA-aptamer which specifically binds to EphA2 and which comprises or consists of sequence SEQ ID NO: 1, wherein optionally one or more of the nucleotides forming the sequence of the aptamer is a modified nucleotide; or a complex comprising said aptamer coupled to a functional substance; or a composition comprising said aptamer or complex. of the invention.

Other objects, features, advantages and aspects of the present application will become apparent to those skilled in the art from the following description and appended claims.

### BRIEF DESCRIPTION OF THE DRAWINGS

FIG. 1A.—Representative western blot showing total EphA2 expression and its phosphorylation at S897 residue in a panel of rhabdomyosarcoma (RMS) cell lines. RH4, RH41, RH28 (expressing low amount of EphA2), RMS13, RH30, CW9019 are ARMS cell lines. RD, RH36, RUCH2, A204 are embryonal RMS cell lines. FIG. 1B.—Representative western blot showing EphA2 expression in a silencing model generated from RH4 cells (RH4shE2 and RH4shE17), in RH4 cells and in RH4/CMV (positive control of silencing). FIG. 1c.—Graphic representation of the results of the migration assay in Boyden chambers using the EphA2 silenced model. RH4/SCR stands for RH4 cells treated with scramble aptamer (an unspecific RNA sequence).

FIG. 2.—Graphic representation of the quantification by qPCR of internalized RNAs after the indicated time points (6, 24, 48 and 72 hours).

FIG. 3A.—Photograph of A673 cell colonies 14 days after scramble aptamer treatment. FIG. 3B.—Photograph of A673 cell colonies 14 days after EphA2 aptamer treatment. FIG. 3C.—Graphic showing the number of colonies as a median percentage counted in each cell line (×3) for A673 (A6) and TC252 (TC2), RH4 and RMS13 treated with either scramble (SCR) or EphA2 aptamer (EPH) at 100 nM every 3 days for 14 days. A673 and TC252 are ES cell lines.

FIG. 4A and FIG. 4B.—show micrographs of A673 migrated cells after scramble and EphA2 aptamer treatment, respectively. Cells were treated with either scramble or EphA2 aptamer 6 hours before placing them at the Boyden chamber at 250 nM once. Micrographs were taken at 48 hours after seeding. FIG. 4C.—Migrated cells were measured at 48 hours (A673, represented as A6 in the graphic) and 6 hours (RMS13). The graphic represents the percentage of migrated cells in the abscise axis.

FIG. 5.—Kaplan-Meier curve comparing differential survival (measured as time to reach enough tumor volume for surgery) of A673 cells growing in the gastrocnemius of mice treated with scramble (n=8, continuous line) or EphA2 aptamer (n=9, dashed line). Long-rank (Mantel-Cox test) analysis was used to generate p-values. P=0.0237.

FIG. 6A.—Micrograph representative of a lung micrometastasis in scramble-treated mice. FIG. 6B.—Micrograph representative of a healthy lung from EphA2 aptamer-treated mice. FIG. 6C.—Quantification of metastases in all the 17 mice: scramble-treated mice (SCR, n=8) and EphA2 aptamer-treated mice (APT, n=9).

FIG. 7.—Graphic representing the EWS/FLI1 expression measured by qPCR. A673 cells (A6) were treated for 48 h with a non-targeting chimera (NT chimera) or the specific chimera (Apt-siEF) at different concentrations (2 μM and 3 μM) without using any lepidic system.

FIG. 8A.—Representation of the secondary structure of the aptamer of sequence SEQ ID NO 2 or 4, predicted using VARNA 3.7. The part marked with the dashed line rectangle corresponds to what it is considered the functional loop, and corresponds to SEQ ID NO 1 or 3, respectively. FIG. 8B.—Model of the secondary structure of an aptamer-siRNA complex. The complex consists of two strands of which the shorter strand (comprising the siRNA guide strand sequence—depicted as open circles) is reverse complementary to the 3′ terminal region of the longer strand (dark grey circles). The longer strand includes the aptamer sequence as well as the sense (passenger, black circles) part of the siRNA, both separated by a 3 nucleotides linker (UUU, light grey). To ease the representation, the aptamer is not the one of panel A.

FIG. 9.—Model of the main hypothesis of the present invention. In the figure, insert shows how the aptamer-siRNA chimera recognizes the receptor in the plasmatic membrane and enters the cell. On the right, a cartoon simulating the structure of the aptamer-siRNA chimera (complex according to the invention).

FIG. 10.—Photograph of A673 cell colonies 14 days after scramble aptamer-EWS/FLI1 siRNA chimera treatment (upper well) and after EphA2-EWS/FLI1 siRNA chimera treatment (lower well).

### DETAILED DESCRIPTION OF THE INVENTION

It must be noted that as used in the present application, the singular forms, e.g., “a”, “an” and “the”, include their correspondent plurals unless the context clearly dictates otherwise.

Unless defined otherwise, all technical and scientific terms used herein have the same meaning as commonly understood by one of ordinary skill in the art to which this invention belongs.

To facilitate understanding and clarify the meaning of specific terms in the context of the present invention, the following definitions and particular and preferred embodiments thereof, applicable to all the embodiments of the different aspects of the present invention, are provided:

The term “aptamer” as used herein refers in general to either an oligonucleotide of a single defined sequence or a mixture of said oligonucleotides, wherein the mixture retains the properties of binding specifically to EphA2. As used herein, “aptamer” refers to single stranded nucleic acid. Structurally, the aptamers of the present disclosure are specifically binding oligonucleotides.

The term “oligonucleotide” as used herein is generic to polydeoxyribonucleotides (containing 2′-deoxy-D-ribose or modified forms thereof), i.e. DNA, to polyribonucleotides (containing D ribose or modified forms thereof), i.e. RNA, and to any other type of polynucleotide which is an N-glycoside or C-glycoside of a purine or pyrimidine base, or modified purine or pyrimidine base or a basic nucleotide. According to the present disclosure the term “oligonucleotide” includes not only those with conventional bases, sugar residues and inter-nucleotide linkages, but also those that contain modifications of any or all of these three moieties (hereinafter also referred as “modified nucleotides”).

The term “RNA-aptamer” as used herein is an aptamer comprising ribonucleoside units, such as adenosine, guanosine, 5-methyluridine, uridine, 5-methylcytidine, cytidine, pseudouridine, inosine, N6-methyladenosine, xanthosine, and wybutosine.

As used herein, the term “specifically binds” shall be taken to mean that the RNA aptamer reacts or associates more frequently, more rapidly, with greater duration and/or with greater affinity with a particular cell or substance than it does with alternative cells or substances. For example, an RNA aptamer that specifically binds to a target protein binds that protein or an epitope or immunogenic fragment thereof with greater affinity, avidity, more readily, and/or with greater duration than it binds to unrelated protein and/or epitopes or immunogenic fragments thereof. It is also understood by reading this definition that, for example, a RNA aptamer that specifically binds to a first target may or may not specifically bind to a second target. As such, “specific binding” does not necessarily require exclusive binding or non-detectable binding of another molecule, this is encompassed by the term “selective binding”. Generally, but not necessarily, reference to binding means specific binding.

The aptamer of the invention is characterized by its capacity to bind EphA2. The capacity of an aptamer to bind to EphA2 can be determined by means of any suitable method which allows determining the binding between two molecules. In one embodiment, the capacity of the aptamer to bind EphA2 is determined by contacting EphA2-expressing cells with the aptamer which has been previously immunofluorescence labelled. If the fluorescence signal is located within the cell, this would be indicative that the aptamer bound to the EphA2 and was subsequently internalized. In an alternative embodiment, the EphA2-expressing cells are contacted with the aptamer and, after a period of time, it is determined the amount of RNA-aptamer within the cells by RT-PCR, using primers amplifying the aptamer sequence (such as those used in Example 3, SEQ ID NO: 24 and 25).EPH receptor A2 (ephrin type-A receptor 2) is a protein that in humans is encoded by the EPHA2 gene. This gene belongs to the ephrin receptor subfamily of the protein-tyrosine kinase family. EPH and EPH-related receptors have been implicated in mediating developmental events, particularly in the nervous system. Receptors in the EPH subfamily typically have a single kinase domain and an extracellular region containing a Cys-rich domain and 2 fibronectin type III repeats. The ephrin receptors are divided into two groups based on the similarity of their extracellular domain sequences and their affinities for binding ephrin-A and ephrin-B ligands. This gene encodes a protein that binds ephrin-A ligands. Uniprot Accession number for human receptor: P29317.

The term “coupled to” as used herein is intended to encompass any construction whereby the RNA aptamer is linked, attached or joined to a functional substance as described herein. Methods for effecting coupling will be known to the skilled in the art and include, but are not limited to conjugation, linking via peptide linker or by direct chemical synthesis of the RNA and functional substance as a whole chain.

As used herein, the term “treat” or “treatment” or “treating” shall be understood to mean administering a therapeutically effective amount of RNA aptamer, complex or composition as disclosed herein and reducing or inhibiting at least one symptom of a clinical condition associated with or caused by cancer.

As used herein, the term “prevent” or “preventing” or “prevention” shall be taken to mean administering a prophylactically effective amount of RNA aptamer, complex or composition according to the present invention and stopping or hindering or delaying the development or progression of at least one symptom of cancer.

The expression “therapeutically effective amount” refers to sufficient quantity of RNA aptamer, complex or composition according to the present invention to reduce or inhibit the number of EphA2 expressing cancer cells and/or one or more symptoms of cancer. The skilled person will be aware that such an amount will vary depending upon, for example, the particular subject and/or the type or severity or level of disease. The term is not be construed to limit the present disclosure to a specific quantity of RNA aptamer, complex or composition.

The expression “prophylactically effective amount” refers to sufficient quantity of RNA aptamer, complex or composition according to the present invention to stop or hinder or delay the development or progression of at least one symptom of cancer. The skilled person will be aware that such an amount will vary depending upon, for example, the particular subject and/or the type or severity or level of disease. The term is not be construed to limit the present disclosure to a specific quantity of RNA aptamer, complex or composition.

As used herein, the term “subject” shall be taken to mean any subject, including a human or non-human subject. The non-human subject may include non-human primates, ungulate (bovines, porches, ovines, caprines, equines, buffalo and bison), canine, feline, lagomorph (rabbits, hares and pikas), rodent (mouse, rat, guinea pig, hamster and gerbil), avian, and fish. Preferably, the subject is a human.

As used herein, the expression “cancer characterised by expressing EphA2” or “EphA2 expressing cancer” refers to a tumor or cancer comprising cells expressing EphA2 (EphA2 positive cells). More particularly, it refers to a cancer over-expressing EphA2, i.e. with cells over-expressing EphA2. It is well-understood by the skilled person in the art which cancers are embraced by the expression “cancer characterised by expressing EphA2” or “EphA2 expressing cancer” (Zhou Y. et al., “Emerging and Diverse Functions of the EphA2 Noncanonical Pathway in Cancer Progression”, Biol. Pharm. Bull. 40, 1616-1624 (2017)).

The term “EphA2+” or “EphA2 expressing cell” as used herein may be used interchangeably. The term encompasses cell surface expression of EphA2 which can be detected by any suitable means.

Several unique properties of aptamers make them attractive tools for use in a wide array of molecular biology applications, and as potential pharmaceutical agents. As therapeutic reagents, RNA aptamers have several advantages over small molecule inhibitors or protein-based reagents. Unlike most small molecule inhibitors, aptamers are highly specific and can be used for targeted therapy. Binding sites for aptamers include clefts and grooves of target molecules resulting in antagonistic activity very similar to many currently available pharmaceutical agents. Moreover, aptamers are structurally stable across a wide range of temperature and storage conditions. In contrast to antibodies, aptamers can be readily chemically synthesized and are amenable to chemical modifications that make them resistant to nucleases and improve their pharmacokinetics in vivo. In addition, chemically modified RNA aptamers have little-to-no immunogenicity and are thus much safer for clinical applications. Given their properties, RNA aptamers are quickly emerging as powerful new therapeutic tools.

Surprisingly, the authors of the present invention have developed a RNA aptamer which binds specifically to EphA2, i.e. a RNA aptamer binding specifically to EphA2, and is able, not only to internalize EphA2 positive cells, but also to exert a therapeutic effect by its own, as it has been explained in detail above.

Thus, in a first aspect, the present invention refers to an RNA-aptamer which specifically binds to EphA2, which:
(i) consists of sequence SEQ ID NO: 1; or, alternatively,
(ii) consists of sequence SEQ ID NO: 1 and the pyrimidine of at least one of the nucleotides forming the sequence is a substituted pyrimidine; or, alternatively,
(iii) comprises the sequence SEQ ID NO: 1, and the pyrimidine of at least one of the nucleotides forming the sequence is a substituted pyrimidine;
   wherein the term “substituted pyrimidine” means that the hydrogen radical of at least one of the carbon or nitrogen atoms forming the pyrimidine ring of formula (I) when the nucleotide is a cytosine, or of formula (II) when the nucleotide is an uracil:

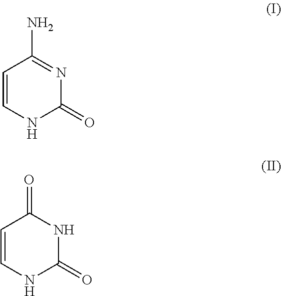

is substituted by a radical other than hydrogen.

The invention also provides a RNA aptamer which binds specifically to EphA2 and which
(i) consists of sequence SEQ ID NO: 1 (gucgucuugcguccccagacgacuc); or
(ii) comprises sequence SEQ ID NO: 2 (gggaggacgaugcgguccuugucgucuugcguccccagacgacucgcccga), optionally comprising one, two or three substitutions located within any of the positions 1-20 and 46-51 of sequence SEQ ID NO: 2.

Particularly, the aptamer is an isolated aptamer. In a particular embodiment, the present invention also provides an isolated RNA aptamer having substantially the same ability to bind to EphA2 as that of an aptamer as defined in the present invention.

In a particular embodiment, the sequence length of the aptamer is between 25 and 100 bases, preferably between 25 and 70 bases and more preferably between 25 and 55 bases, enabling easy chemical synthesis. The term “base” can be interchangeably used by “ribonucleoside unit” or “nucleotide base” or “residue” such as guanine (G), adenine (A), uracil (U) or cytosine (C). The bases may form hydrogen bonds between cytosine and guanine, adenine and uracil and between guanine and uracil.

In an embodiment, the aptamer comprises the sequence SEQ ID NO: 2.

The aptamer of the present invention can be synthesised by any method known in the art. In a preferred embodiment, the aptamer is produced by cell-SELEX (Systematic Evolution of Ligands by EXponential Enrichment), more preferably produced by the method herein described (see Example 1). Advantageously, the cell-SELEX method allows for the generation of aptamers against cell surface targets by replicating the native conformation and glycosylation pattern of the extracellular regions of proteins. Thus, the aptamer will bind to EphA2 in a cellular context and internalize into EphA2 expressing cells (i.e. EphA2 positive cells).

One potential problem encountered in the use of nucleic acids as therapeutics is that oligonucleotides in their phosphodiester form may be quickly degraded in body fluids by intracellular and extracellular enzymes such as endonucleases and exonucleases before the desired effect is manifest. It is well-known in the state of the art that an aptamer may comprise one or more modifications (modified aptamer) that improve aptamer stability (in vitro or in vivo), e.g., modifications to make the aptamer resistant to nucleases. Modifications to generate oligonucleotides which are resistant to nucleases are well-known to those skilled in the art and can include one or more substitute internucleotide linkages, altered sugars, altered bases, or combinations thereof. Such modifications, giving rise to “modified nucleotides”, include 2′-position sugar modifications, 2′-position pyrimidine modifications, 5-position pyrimidine modifications, 8-position purine modifications, modifications at exocyclic amines, substitution of 4-thiouridine, substitution of 5-bromo or 5-iodo-uracil, backbone modifications, phosphorothioate or (C₁-C₁₀)alkyl phosphate modifications, methylations, and unusual base-pairing combinations such as the isobases isocytidine and isoguanosine; 3′ and 5′ modifications such as capping; conjugation to a high molecular weight, non-immunogenic compound; conjugation to a lipophilic compound; and phosphate backbone modification.

In a particular embodiment of the invention, the “modified nucleotide” is a modified cytosine or uracil. In another embodiment, the “modified nucleotide” is a cytosine or uracil wherein the pyrimidine moiety is a “modified pyrimidine”, as defined above.

In the present invention, the aptamer of the first aspect includes at least one substituted pyrimidine. The RNA oligonucleotides can include two types of pyrimidine derivatives:

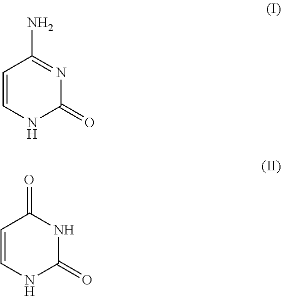 [SMILES: Nc1cc[nH]c(=O)n1 ; O=c1cc[nH]c(=O)[nH]1]

Unless otherwise stated, when reference is made in the present invention to a “substituted pyrimidine” it is to be understood as the pyrimidine of formula (I) or (II) wherein at least one of the hydrogen radicals bound to at least one of the carbon or nitrogen atoms forming part of the pyrimidine ring, has been replaced by a different radical.

In one embodiment, the RNA-aptamer comprises or consists of SEQ ID NO: 2, and the pyrimidine of at least one of the nucleotides forming the sequence is a substituted pyrimidine. In another embodiment, the RNA-aptamer of the invention consists of sequence SEQ ID NO:2 and the pyrimidine of at least one of the nucleotides forming the sequence is a substituted pyrimidine.

In another embodiment, at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99% of the pyrimidines are substituted pyrimidines. Particularly, all the pyrimidines of the nucleotide sequence are substituted pyrimidines.

In another embodiment, the one or more substituted pyrimidine(s) are pyrimidines of formula (I) or (II) comprising a radical other than hydrogen at 2′-position. The term “comprising one radical other than hydrogen at 2′-position” means that the pyrimidine moiety includes at position 2′ a radical other than hydrogen, without excluding the possibility that the pyrimidine ring can include further substitutions in other positions of the ring.

In another embodiment, the one or more substituted pyrimidine(s) consist(s) of 2′-substituted pyrimidine(s). The term “consist(s) of 2′-substituted pyrimidine(s)” means that the pyrimidine moiety show a single modification (i.e., substitution by a radical other than hydrogen) only at 2′-position, and further substitutions in other positions of the ring are excluded.

In another embodiment, the aptamer of the invention includes one or more substituted pyrimidines comprising one radical other than hydrogen in 2′-position and one or more substituted pyrimidines consisting of 2′-substituted pyrimidines, as defined above.

In another embodiment, the aptamer of the invention only includes substituted pyrimidines consisting of 2′-substituted pyrimidines, as defined above.

In another embodiment, the aptamer comprises two or more substituted pyrimidines, as defined above, and the radical other than hydrogen is the same in all the substituted pyrimidines.

In another embodiment, the radical other than hydrogen is selected from halogen, —NR₁R₂, —O—(C₁-C₆)alkyl, —SR₃, azide, and (C₁-C₆)alkyl optionally substituted by —OH, wherein R₁, R₂ and R₃ are selected from —H, (C₁-C₆)alkyl, and (C₁-C₆)alkenyl. In another embodiment the radical other than hydrogen is halogen, particularly fluoride.

The term (C₁-C₆)alkyl refers to a saturated straight or branched alkyl chain having from 1 to 6 carbon atoms. Illustrative non-limitative examples are: methyl, ethyl, propyl, isopropyl, butyl, isobutyl, sec-butyl, tert-butyl, n-pentyl, neo-pentyl and n-hexyl.

The term (C₂-C₆)alkenyl refers to a saturated straight, or branched alkyl chain containing from 2 to 6 carbon atoms and also containing one or more double bonds. Illustrative non-limitative examples are ethenyl, propenyl, butenyl, 1-methyl-2-buten-1-yl, and the like.

The term “halogen” refers to the group in the periodic table consisting of five chemically related elements: fluorine (F), chlorine (Cl), bromine (Br), iodine (I), and astatine (At).

In another particular embodiment, the aptamer is modified by coupling the 5′-end and/or 3′-end to a fluorophore or inverted dT or to a polyalkylene glycol, preferably polyethylene glycol (PEG) molecule.

Particularly, when the modification is performed by modified nucleosides (e.g., 2′-fluoro-pyrimidines), the aptamer is highly resistant to nuclease-mediated degradation and can thus be used in cell culture as well as in animals/subjects. In another preferred embodiment, the pyrimidine bases are 2′-fluoro (2′-F) modified, more preferably as indicated in any one of sequences SEQ ID NO 3 (gUCgUCUUgCgUCCCCagaCgaCUC, capital letter denoting 2′F-modified base) and SEQ ID NO 4 (gggaggaCgaUgCggUCCUUgUCgUCUUgCgUCCCCagaCgaCUCgCCCga, capital letter denoting 2′F-modified base). Thus, in preferred embodiments the aptamer (i) consists of sequences SEQ ID NO 3; or (ii) comprises or consists or consists essentially of sequence 4, optionally comprising one, two or three substitutions located within any of the positions 1-20 and 46-51 of sequence SEQ ID NO: 4. More preferably, it comprises SEQ ID NO 4 which, as shown in the Examples, specifically binds and internalizes EphA2 positive cells and it is a successful delivery agent of functional substances (e.g., siRNA).

Particularly, when modification is performed by terminal addition of PEG, the molecular weight of PEG is not particularly limited, and is preferably 1000-100000, more preferably 20000-90000. PEG may be linear or branched into two or more chains (multi-arm PEG). As for terminal addition of PEG, it may be added to only one of the 3′-end and 5′-end, or both of 3′-end and 5′-end. Preferably, PEG is added to the 5′end of the aptamer. Advantageously, this keeps the aptamer in the bloodstream and is not filtered by the kidney.

Such PEG is not particularly limited, and those skilled in the art can appropriately select and use commercially available or known PEG. In the present invention, PEG may be directly added to the terminus. It is more preferable that a linker having a group which can bind to PEG and the like should be added to the terminus thereof, and PEG should be added to the aptamer provided herein via the linker. As PEG and linker, commercially available products can be preferably used. The reaction conditions and the like relating to the binding of PEG, a linker and the aptamer provided herein can be appropriately determined by those skilled in the art.

In another embodiment, the aptamer of the invention is selected from SEQ ID NO:1, SEQ ID NO: 3 and SEQ ID NO: 4.

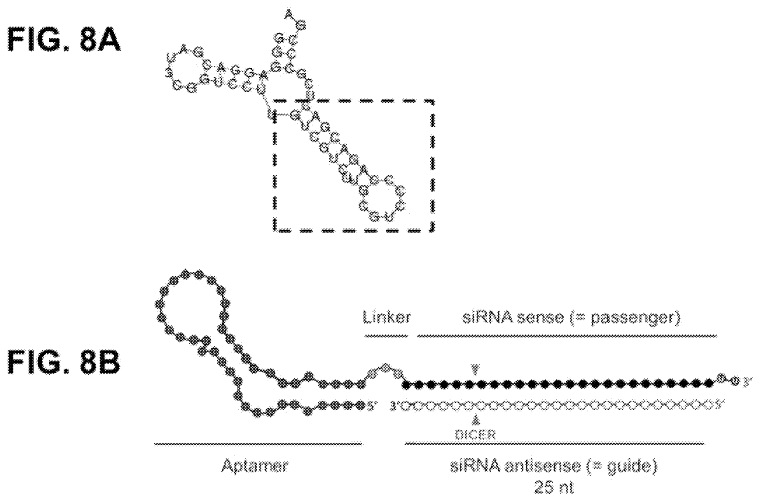

Aptamer binding is highly dependent on the secondary structure formed by the aptamer oligonucleotide. The secondary structures of the RNA strand of the aptamer of the invention were predicted using VARNA 3.7. The predicted secondary structure of the aptamer of SEQ ID NO: 2 or 4 is shown in FIG. 8A. It can be seen that the predicted secondary structure has a loop, which has sequence SEQ ID NO: 1 or 3 (dashed line rectangle in FIG. 8A). While not wishing to be bound by theory, the inventors consider that this loop is a functional loop, loop binding to the receptor, so the aptamer consisting of this sequence can be functional and specific for EphA2. In a particular embodiment, the aptamer of the invention has the secondary structure shown in FIG. 8A.

An aptamer binds to the target molecule in a wide variety of binding modes, such as ionic bonds based on the negative charge of the phosphate group, hydrophobic bonds and hydrogen bonds based on ribose, and hydrogen bonds and stacking interaction based on nucleic acid bases. In particular, ionic bonds based on the negative charge of the phosphate group, which are present in the same number as the number of constituent nucleotides, are strong, and bind to lysine and arginine being present on the surface of the positive charge of protein. For this reason, nucleic acid bases not involved in the direct binding to the target molecule can be substituted. In particular, because the region of stem structure has already formed base pairs and faces the inside of the double helical structure, nucleic acid bases are unlikely to bind directly to the target molecule. Therefore, even when a base pair is replaced with another base pair, the activity of the aptamer often does not decrease. Thus, as defined above, the aptamer of the present invention can comprise one, two or three substitutions outside the predicted functional loop, that is, at any position within positions 1-20 and 46-51 of SEQ ID NO 2 or SEQ ID NO 4.

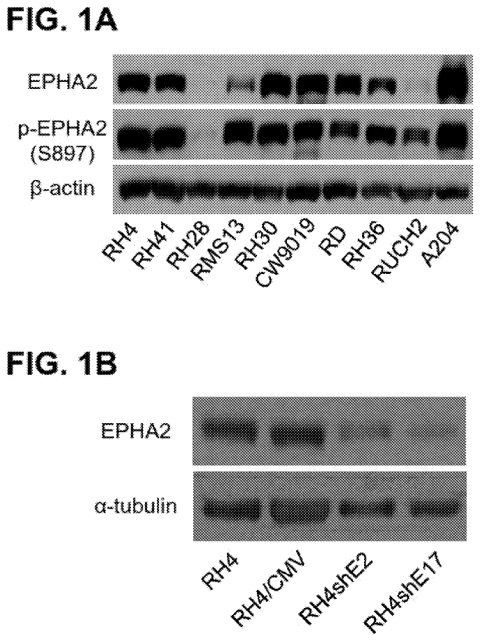

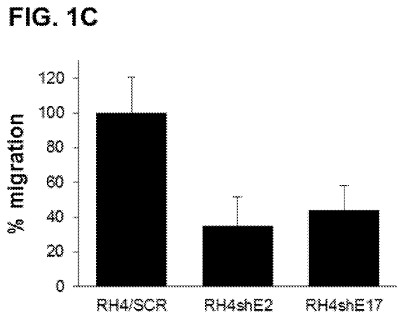

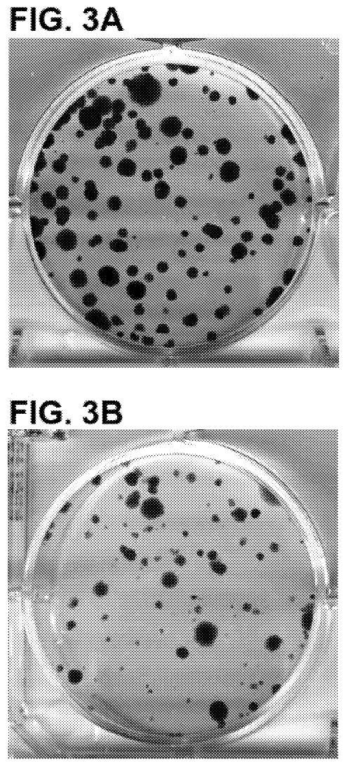

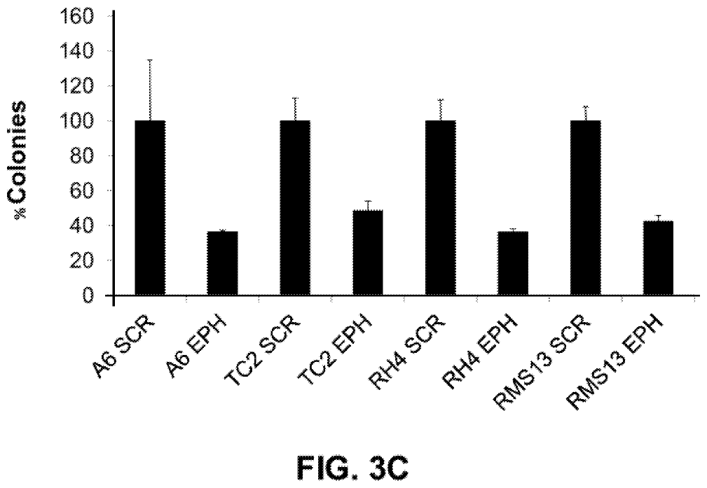

Regarding modifications of the 2′-position of ribose, the functional group at the 2′-position of ribose infrequently interacts directly with the target molecule, but in many cases, it is of no relevance, and can be substituted by another modified molecule. In another particular embodiment according to any one of the preceding embodiments, the aptamer specifically binds to EphA2 positive (cancer) cell(s). This is shown, for example, in Example 3 wherein an aptamer of SEQ ID NO 4 specifically binds to ES cells. In said Example it is also shown that the aptamer is able to internalize said EphA2 positive cells. Since cell migration and colony formation are blocked in RMS cells with stable knockdown of EphA2 (see FIGS. 1C, 3A, 3B, and 3C), it is expected that the aptamer internalizes EphE2 positive RMS cells, as it does in ES cells. Thus, in a particular embodiment, the aptamer internalizes EphA2 positive (cancer) cell(s), and, therefore, it can be used as delivery system for said specific cells.

The aptamer of the present invention can be coupled to a functional substance forming a complex (also referred to as chimera hereinafter). Like this, the aptamer not only provides a therapeutic effect but also acts as a delivery agent of the functional substance to EphA2 positive cancer cells. Thus, in a second aspect, the present invention refers to a complex comprising the RNA-aptamer according to any one of the embodiments of the first aspect of the invention, coupled to a functional substance.

All the embodiments provided above regarding the aptamer are also embodiments of the second aspect of the invention.

The coupling between the aptamer and the functional substance in the complex can be a covalent bond or a non-covalent bond. The complex of the present invention can be one wherein the aptamer of the present invention and one or more (e.g., 2 or 3) of functional substances of the same kind or different kinds are bound together.

Preferably, the functional substance is coupled to the 3′-end of the aptamer.

In a particular embodiment of the complex according to any one of the preceding embodiments, the functional substance is coupled to the aptamer by a spacer or linker, preferably of 2-5 nucleotides, more preferably of 3 nucleotides (e.g., UUU), and/or the functional substance comprises a tail at its 3′-end, preferably a tail of 2-5 nucleotides, more preferably of 2 or 3 nucleotides (e.g., UU or UUU). Advantageously, this linker and/or tail improves stability of the complex.

In one embodiment of the complex of the invention the spacer comprises one or more uracil nucleotides. In another embodiment, the spacer is made of uracil nucleotides. In another embodiment, the spacer consists of 2-5 uracil nucleotides, particularly 2-3 uracil nucleotides, more particularly 3 uracil nucleotides.

The functional substance is not particularly limited, as far as it newly adds a certain function to the aptamer of the present invention, or is capable of changing (e.g., improving) a certain characteristic which an aptamer of the present invention can possess. As examples of the functional substance, proteins (such as ribozyme), peptides, amino acids, lipids, sugars, monosaccharides, polynucleotides, and nucleotides can be mentioned. As further examples of the functional substance, affinity substances (e.g., biotin, streptavidin, polynucleotides possessing affinity for target complementary sequence (such as siRNA, microRNA (also referred to as miR, mir, or miRNA), shRNA), antibodies, glutathione Sepharose, histidine), substances for labeling (e.g., fluorescent substances, luminescent substances, radioisotopes), enzymes (e.g., horseradish peroxidase, alkaline phosphatase), drugs (e.g., chemotherapeutic agents such as doxorubicin, gemcitabine, etc.) can be mentioned.

In a particular embodiment of the complex of the second aspect of the invention, the functional substance is:
(i) an siRNA, microRNA, shRNA or a ribozyme, preferably siRNA or microRNA; or
(ii) a moiety selected from a radionuclide, a chemotherapeutic agent and combinations thereof, preferably a chemotherapeutic agent.

Preferably, the functional substance is siRNA, microRNA, a chemotherapeutic agent or a combination of siRNA or miRNA and a chemotherapeutic agent. The complex, either with siRNAs or miRNAs, loaded with small amounts of chemotherapy molecules reduces adverse effects of the chemotherapeutic agent.

In another embodiment, the functional substance is a siRNA or miRNA and comprises a nucleotide tail at its 3′-end, preferably a tail made of 2-5 nucleotides, more preferably of 2 or 3 nucleotides. In another embodiment, optionally in combination with any of the embodiments provided above or below, the functional substance is a siRNA or miRNA and it comprises a 3′-end tail comprising one or more uracil nucleotides. In another embodiment the functional substance is a siRNA or miRNA and it comprises a 3′-end tail consisting of 2-5 uracil nucleotides, particularly 3 nucleotides.

In another embodiment, the complex comprises the aptamer of the invention coupled through a spacer, made of 2-5 nucleotides, to a miRNA or siRNA which comprises a 3′-end tail made of 2-5 nucleotides. In another embodiment, the complex comprises the aptamer of the invention coupled through a spacer, made of 2-5 uracil nucleotides, to a miRNA or siRNA which comprises a 3′-end tail made of 2-5 uracil nucleotides. In another embodiment, the complex comprises the aptamer of the invention coupled through a spacer, made of 2-5 uracil nucleotides, to a siRNA which comprises a 3′-end tail made of 2-5 uracil nucleotides. In another embodiment, the complex of the invention comprises the aptamer herein provided coupled through a spacer made of 2-3 nucleotides to a miRNA or siRNA comprising a 3′-end tail made of 2-3 nucleotides. In another embodiment, the complex of the invention comprises the aptamer herein provided coupled through a spacer made of 2-3 uracil nucleotides to a miRNA or siRNA comprising a 3′-end tail made of 2-3 uracil nucleotides. In another embodiment, the complex of the invention comprises the aptamer herein provided coupled through a spacer, made of 2-3 nucleotides, to a siRNA which comprises a 3′-end tail made of 2-3 nucleotides. In another embodiment, the complex of the invention comprises the aptamer herein provided coupled, through a spacer made of 2-3 uracil nucleotides, to a siRNA comprising a 3′-end tail made of 2-3 uracil nucleotides.

In another embodiment, the complex comprises the aptamer comprising or consisting of sequence SEQ ID NO: 2, wherein all the pyrimidines are substituted pyrimidines, preferably 2′-substituted pyrimidines, coupled through a spacer, made of 2-5 nucleotides, to a miRNA or siRNA which comprises a 3′-end tail made of 2-5 nucleotides. In another embodiment, the complex comprises the aptamer comprising or consisting of sequence SEQ ID NO: 2, wherein all the pyrimidines are substituted pyrimidines, preferably 2′-substituted pyrimidines, coupled through a spacer, made of 2-5 uracil nucleotides, to a miRNA or siRNA which comprises a 3′-end tail made of 2-5 uracil nucleotides. In another embodiment, the complex comprises the aptamer comprising or consisting of sequence SEQ ID NO: 2, wherein all the pyrimidines are substituted pyrimidines, preferably 2′-substituted pyrimidines, coupled through a spacer, made of 2-5 uracil nucleotides, to a siRNA which comprises a 3′-end tail made of 2-5 uracil nucleotides. In another embodiment, the complex of the invention comprises the aptamer comprising or consisting of sequence SEQ ID NO: 2, wherein all the pyrimidines are substituted pyrimidines, preferably 2′-substituted pyrimidines, coupled through a spacer made of 2-3 nucleotides to a miRNA or siRNA comprising a 3′-end tail made of 2-3 nucleotides. In another embodiment, the complex of the invention comprises the aptamer comprising or consisting of sequence SEQ ID NO: 2, wherein all the pyrimidines are substituted pyrimidines, preferably 2′-substituted pyrimidines, coupled through a spacer made of 2-3 uracil nucleotides to a miRNA or siRNA comprising a 3′-end tail made of 2-3 uracil nucleotides. In another embodiment, the complex of the invention comprises the aptamer comprising or consisting of sequence SEQ ID NO: 2, wherein all the pyrimidines are substituted pyrimidines, preferably 2′-substituted pyrimidines, coupled through a spacer, made of 2-3 nucleotides, to a siRNA which comprises a 3′-end tail made of 2-3 nucleotides. In another embodiment, the complex of the invention comprises the aptamer comprising or consisting of sequence SEQ ID NO: 2, wherein all the pyrimidines are substituted pyrimidines, preferably 2′-substituted pyrimidines, coupled, through a spacer made of 2-3 uracil nucleotides, to a siRNA comprising a 3′-end tail made of 2-3 uracil nucleotides.

In another embodiment, the complex comprises the aptamer comprising or consisting of sequence SEQ ID NO: 4, coupled through a spacer, made of 2-5 nucleotides, to a miRNA or siRNA which comprises a 3′-end tail made of 2-5 nucleotides. In another embodiment, the complex comprises the aptamer comprising or consisting of sequence SEQ ID NO: 4, coupled through a spacer, made of 2-5 uracil nucleotides, to a miRNA or siRNA which comprises a 3′-end tail made of 2-5 uracil nucleotides. In another embodiment, the complex comprises the aptamer comprising or consisting of sequence SEQ ID NO: 4, coupled through a spacer, made of 2-5 uracil nucleotides, to a siRNA which comprises a 3′-end tail made of 2-5 uracil nucleotides. In another embodiment, the complex of the invention comprises the aptamer comprising or consisting of sequence SEQ ID NO: 4, coupled through a spacer made of 2-3 nucleotides to a miRNA or siRNA comprising a 3′-end tail made of 2-3 nucleotides. In another embodiment, the complex of the invention comprises the aptamer comprising or consisting of sequence SEQ ID NO: 4, coupled through a spacer made of 2-3 uracil nucleotides to a miRNA or siRNA comprising a 3′-end tail made of 2-3 uracil nucleotides. In another embodiment, the complex of the invention comprises the aptamer comprising or consisting of sequence SEQ ID NO: 4, coupled through a spacer, made of 2-3 nucleotides, to a siRNA which comprises a 3′-end tail made of 2-3 nucleotides. In another embodiment, the complex of the invention comprises the aptamer comprising or consisting of sequence SEQ ID NO: 4, coupled, through a spacer made of 2-3 uracil nucleotides, to a siRNA comprising a 3′-end tail made of 2-3 uracil nucleotides.

In a preferred embodiment according to any one of the preceding embodiments, the functional substance is siRNA. Preferably the siRNA consists of 20-30 nucleotides, more preferably 23-27 nucleotides and even more preferably consists of 25 nucleotides. Advantageously, these siRNA favor the activity of the dicer complex to release the mature siRNA.

Since RNAi technology is readily adaptable to inhibit the expression of virtually any gene in the human genome, it has become a valuable tool for elucidating mechanisms of deregulated cell growth and survival during malignancy. Furthermore, its potential use as a cancer therapeutic tool has also become apparent and highly pursued. However, despite the development of several effective anticancer cell siRNAs, to date there are no approved siRNA-based therapies for the treatment of cancer. The major problem for the successful translation of siRNAs into effective therapies for use in the clinic is delivery and safety (due to toxicity problems). Surprisingly, the authors of the present invention have developed an aptamer that, when linked to a siRNA, serves as delivery agent into EphA2 positive cells. Moreover, the siRNA is protected against degradation and it is correctly processed by DICER, resulting in silencing of the target gene of said siRNA (see Example 8).

As mentioned earlier, TAS are characterised by the unique presence of a specific fusion protein due to a tumor-specific chromosomal translocation. Thus, in a preferred embodiment according to the previous paragraph, the siRNA is directed against the specific translocation product characterising the EphA2 expressing cancer, such as TAS. For example, it is directed to EWS/FLI1, the specific translocation product characterising ES, to PAX3/FOXO1 the specific translocation product characterising ARMS, to SS18/SSX1-2 the specific translocation products characterising SS, to CIC/DUX4 and BCOR-CCNB3 specific translocation product characterising Ewing-like sarcomas, to EWS/WT1 specific translocation product characterising desmoplastic small round cell tumor (DSRCT) and, to EWS/DDIT3 and FUS/DDIT3 specific translocation products characterizing Myxoid Liposarcoma (MLS).

In a preferred embodiment according to any one of the preceding embodiments, the aptamer is coupled to a siRNA and said siRNA comprises or consists of any one of sequences SEQ ID NO 5 (cgggcagcagaacccuucuuaugac), SEQ ID NO 6 (auggccucucaccucagaauucaau) and SEQ ID NO 7 (ugcccaagaagccagcagaggaauu). Each one of these sequences is specific for the chromosomal translocation characterising ES, ARMS and SS, respectively. More preferably, the complex of the invention comprises or consists of a sequence selected from:

SEQ ID NO 11:
gggaggacgaugcgguccuugucgucuugcguccccagacgacucgcccg
auuucgggcagcagaacccuucuuaugacuu,
SEQ ID NO 12:
gucgucuugcguccccagacgacucuuucgggcagcagaacccuucuuau
gacuu,
SEQ ID NO 13:
gggaggacgaugcgguccuugucgucuugcguccccagacgacucgcccg
auuuauggccucucaccucagaauucaauuu,
SEQ ID NO 14:
gucgucuugcguccccagacgacucuuuauggccucucaccucagaauuc
aauuu,
SEQ ID NO 15:
gggaggacgaugcgguccuugucgucuugcguccccagacgacucgcccg
auuuugcccaagaagccagcagaggaauuuu,
and
SEQ ID NO 16:
gucgucuugcguccccagacgacucuuuugcccaagaagccagcagagga
auuuu.

These complexes have the aptamer of the invention (SEQ ID NO 3 or 4) linked by a 3 UUU spacer to the siRNA of sequence SEQ ID NO 5, SEQ ID NO 6, or SEQ ID NO 7 with a UU tail at the 3′-end, and are therefore useful as therapeutic agents for ES, ARMS or SS, respectively.

In another preferred embodiment of the complex according to any one of the preceding embodiments, the functional substance is one or various miR(s). Particularly, the miRNA is a tumor suppressor (onco-suppressor) miRNA, more particularly a tumor suppressor miRNA of a tumor characterised by expressing EphA2. In a preferred embodiment, the miRNA is selected from the group consisting of mir-130a (tumor suppressor in prostate cancer); mir-143 (tumor suppressor in osteosarcoma and SS); mir-145 (tumor suppressor in ES, osteosarcoma, prostate, pancreatic, breast and colorectal cancer); mir-302, mir-505 or mir-520c (tumor suppressors in colorectal cancer); mir-202 (tumor suppressor in pancreatic cancer); mir-34a (tumor suppressor in ES and prostate cancer); mir-206 and mir-29 (tumor suppressors in RMS) and mir-424 (tumor suppressor in breast cancer).

Although the creation of aptamer-siRNA or aptamer-miRNA complexes significantly improves siRNA or miRNA biopharmaceutical properties, additional modification might further improve the product. In a recent study, the chemical conjugation of a 20 kDa PEG group extended the circulating half-life of an aptamer-siRNA complex. Such PEG molecule was placed at the siRNA passenger strand by chemical synthesis without affecting binding to aptamer target or target gene silencing activity (Dassie et al.). Thus, in a particular embodiment of the complex of the invention according to any one of the preceding embodiments, the functional substance is siRNA to which a PEG molecule is coupled at the siRNA passenger strand, preferably the PEG is coupled by chemical synthesis.

Moreover, nanotechnology has been shown to prolong the stability of nucleic acids in serum and to enhance tumor distribution of carried agents by the enhanced permeability and retention effect, which consists in the accumulation of nanoparticles in the tumor microenvironment due to the abnormal and leaky tumor vasculature and the absence of tumor lymphatic vessels. PEGylated nanoparticles of biodegradable and FDA-approved polymers, such as poly-lactide-co-glycolide acid (PLGA), increase systemic circulation time and improve tumor distribution as compared to non-PEGylated nanoparticles. In addition, PEG can be used to conjugate targeting molecules to the surface of the nanoparticle (Cheng and Saltzman). Thus, in a particular embodiment according to any one of the preceding embodiments, the complex is in the form of PEGylated nanoparticles carrying PEG-conjugated aptamer-siRNA or miRNA complexes on the surface.

Advantageously, this complex can be formulated without liposomes while protecting the aptamer from its degradation. Not having to formulate the complex within liposomes, has multiple advantages. Amongst others, it prevents the toxicity inherent to liposomes, toxicity that accounts for an increase in cell death of approximately 20%.

In another particular embodiment according to any one of the preceding embodiments, the siRNA or microRNA can comprise modifications to protect them from nuclease degradation. The modifications explained above for the aptamer of the invention are applicable to the siRNA and microRNA. In a particular embodiment, the siRNA or microRNA comprises modified nucleosides (e.g., 2′-fluoro-pyrimidines), like this it is highly resistant to nuclease-mediated degradation and can thus be used in cell culture as well as in animals/subjects. Preferably, one or more of the pyrimidine bases forming part of the miRNA or siRNA are substituted pyrimidines. All the embodiments provided above for “substituted pyrimidines” in aptamers, apply and, therefore are also embodiments, of the “substituted pyrimidines” optionally included in the siRNA or microRNA forming part of the complex of the invention. In one embodiment, all or part of the pyrimidine bases of the miRNA or siRNA are 2′-modified pyrimidines, the radical being selected from halogen, —NR₁R₂, —O—(C₁-C₆)alkyl, —SR₃, azide, and (C₁-C₆)alkyl optionally substituted by —OH, wherein R₁, R₂ and R₃ are selected from —H, (C₁-C₆)alkyl, and (C₁-C₆)alkenyl. In another embodiment, the substituted pyrimidine bases of the miRNA or siRNA are all are 2′-fluoro (2′-F) modified. Like this, in a preferred embodiment the siRNA comprises or consists of sequence SEQ ID NO 8 (CgggCagCagaaCCCUUCUUaUgaC, capital letter denotes 2′-F modified base), SEQ ID NO 9 (aUggCCUCUCaCCUCagaaUUCaaU, capital letter denotes 2′-F modified base) or SEQ ID NO 10 (UgCCCaagaagCCagCagaggaaUU, capital letter denotes 2′-F modified base).

More preferably, the complex comprises or consists of a sequence, in which capital letter denotes 2′-F modified base, selected from:

SEQ ID NO 17:
gggaggaCgaUgCggUCCUUgUCgUCUUgCgUCCCCagaCgaCUCgCCCg
auuuCgggCagCagaaCCCUUCUUaUgaCuu,
SEQ ID NO 18:
gUCgUCUUgCgUCCCCagaCgaCUCuuuCgggCagCagaaCCCUUCUUaU
gaCuu,
SEQ ID NO 19:
gggaggaCgaUgCggUCCUUgUCgUCUUgCgUCCCCagaCgaCUCgCCCg
auuuaUggCCUCUCaCCUCagaaUUCaaUuu,
SEQ ID NO 20:
gUCgUCUUgCgUCCCCagaCgaCUCuuuaUggCCUCUCaCCUCagaaUUC
aaUuu,
SEQ ID NO 21:
gggaggaCgaUgCggUCCUUgUCgUCUUgCgUCCCCagaCgaCUCgCCCg
auuuUgCCCaagaagCCagCagaggaaUUuu,
and
SEQ ID NO 22:
gUCgUCUUgCgUCCCCagaCgaCUCuuuUgCCCaagaagCCagCagagga
aUUuu.

These complexes comprise 2′-fluoro modified pyrimidines in the aptamer and siRNA rendering them resistant to nuclease degradation and are useful as therapeutic agents for ES, ARMS or SS.

In another preferred embodiment of the complex of the invention, the functional substance is a chemotherapeutic agent. In one embodiment, the chemotherapeutic agent is selected from the group consisting of doxorubicin, gemcitabine, docetaxel, trabectedin, temozolomide, eribuline and combinations thereof. More preferably, the chemotherapeutic agent is selected from the group consisting of doxorubicin, gemcitabine, docetaxel and combinations thereof.

In another particular embodiment of the complex according to the present invention, the functional substance is a detectable label, preferably selected from the group consisting of an enzyme, prosthetic group, fluorescent material, luminescent material, bioluminescent material, electron dense label, labels for magnetic resonance imaging, radioactive material, and combinations of these. Like this, the complex can serve as diagnostic agent since it can detect EphA2 positive cells.

The aptamer or complex of the present invention can be used as, for example, in the form of a pharmaceutical composition. Thus, in a third aspect, the present invention refers to a composition comprising the RNA-aptamer or the complex of the invention, at a therapeutically effective amount together with acceptable or pharmaceutical excipients and/or carriers. The expression “excipients and/or carriers” refers to acceptable materials, compositions or vehicles. Each component must be pharmaceutically acceptable in the sense of being compatible with the other ingredients of the composition. It must also be suitable for use in contact with the tissue or organ of humans and non-human animals without excessive toxicity, irritation, allergic response, immunogenicity or other problems or complications commensurate with a reasonable benefit/risk ratio. Examples of suitable acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical or cosmetical composition, its use is contemplated to be within the scope of this invention.

The formulations of the pharmaceutical compositions described herein may be prepared by any method known or hereafter developed in the art of pharmacology. In general, such preparatory methods include the step of bringing the active ingredient (aptamer or complex) into association with a excipient and/or one or more other accessory ingredients, and then, if necessary and/or desirable, shaping and/or packaging the product into a desired single- or multi-dose unit.

A pharmaceutical composition of the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient.

The relative amounts of the active ingredient (aptamer or complex of the invention), the acceptable excipients, and/or any additional ingredients in the composition of the invention will vary, depending upon the identity, size, and/or condition of the subject treated and further depending upon the route by which the composition is to be administered.

Examples of the pharmaceutically acceptable carrier include, but are not limited to, excipients such as sucrose, starch, mannit, sorbit, lactose, glucose, cellulose, talc, calcium phosphate, and calcium carbonate; binders such as cellulose, methylcellulose, hydroxylpropylcellulose, polypropylpyrrolidone, gelatin, gum arabic, polyethylene glycol, sucrose, and starch; disintegrants such as starch, carboxymethylcellulose, hydroxylpropylstarch, sodium-glycol-starch, sodium hydrogen carbonate, calcium phosphate, and calcium citrate; lubricants such as magnesium stearate, Aerosil®, talc, and sodium lauryl sulfate; flavoring agents such as citric acid, menthol, glycyrrhizin-ammonium salt, glycine, and orange powder; preservatives such as sodium benzoate, sodium hydrogen sulfite, methylparaben, and propylparaben; stabilizers such as citric acid, sodium citrate, and acetic acid; suspending agent such as methylcellulose, polyvinylpyrrolidone, and aluminum stearate; dispersant such as surfactants; diluents such as water, saline, and orange juice; base waxes such as cacao butter, polyethylene glycol, and white kerosene; and the like.

The composition of the present invention can be formulated in any form known by the skilled in the art suitable for the desired administration (e.g., oral, parenteral, inhalant).

In a particular embodiment according to any one of the preceding embodiments, the aptamer and/or complex of the composition or medicament of the present invention is/are the active principle(s) of the composition.

The present invention also provides a solid phase carrier having the aptamer or the complex of the present invention immobilized thereon. As examples of the solid phase carrier, a substrate, a resin, a plate (e.g., multiwell plate), a filter, a cartridge, a column, and a porous material can be mentioned. The substrate can be one used in DNA chips, protein chips and the like; for example, nickel-PTFE (polytetrafluoroethylene) substrates, glass substrates, apatite substrates, silicon substrates, alumina substrates and the like, and substrates prepared by coating these substrates with a polymer and the like can be mentioned. As examples of the resin, agarose particles, silica particles, a copolymer of acrylamide and N,N′-methylenebisacrylamide, polystyrene-crosslinked divinylbenzene particles, particles of dextran crosslinked with epichlorohydrin, cellulose fiber, crosslinked polymers of allyldextran and N,N′-methylenebisacrylamide, monodispersed synthetic polymers, monodispersed hydrophilic polymers, Sepharose®, Toyopearl® and the like can be mentioned, and also resins prepared by binding various functional groups to these resins were included. The solid phase carrier of the present invention can be useful in, for example, purifying, detecting and quantifying EphA2. The aptamer or the complex of the present invention can be immobilized onto a solid phase carrier by a method known by the skilled person.

As mentioned earlier the aptamers of the present invention can be used as delivery systems and have diagnostic and therapeutic potential. Thus, in a fourth aspect, the present invention refers to an aptamer, complex or composition according to any one of the embodiments provided above, for use in the treatment or prevention of cancer or cancer metastasis, wherein the cancer is characterised by expressing EphA2 (EphA2 expressing cancer).

All the embodiments provided above for the aptamer, complex and composition of the invention are also embodiments of the fourth aspect of the invention.

The fourth aspect also includes a method of treatment of a cancer or cancer metastasis characterised by expressing EphA2 in a subject, the method comprising the administration to said subject of a therapeutically effective amount of a RNA aptamer, or complex, or composition according to any one of the embodiments provided above.

The fourth aspect also includes a method of prevention of an EphA2 expressing cancer or EphA2 expressing cancer metastasis in a subject, the method comprising the administration to said subject of a prophylactically effective amount of a RNA aptamer, or complex, or composition according to any one of the embodiments provided above.

In an embodiment of the fourth aspect, the present invention provides the combined use of the aptamer, complex or composition as defined herein together with a further anti-cancer substance/therapy in the treatment of cancer or cancer metastasis characterized by expressing EphA2. They can be administered sequentially, simultaneously, together or separately.

In a particular embodiment of the fourth aspect, the aptamer comprises or consists of sequence SEQ ID NO: 1, SEQ ID NO: 2, SEQ ID NO: 3 or SEQ ID NO: 4, and the complex comprises or consists of sequence SEQ ID NO: 17.In a fifth aspect, the present invention refers to the use of the aptamer, or complex, or composition according to any one of the embodiments provided above, for in vitro or ex vivo diagnosis of cancer or cancer metastasis, wherein the cancer is an EphA2 expressing cancer.

The fifth aspect also includes an in vitro method of diagnosis of a cancer or cancer metastasis characterised by expressing EphA2 in a subject, the method comprising contacting the RNA aptamer, or a complex according to any one of the embodiments of the third aspect of the invention with a test sample of the subject.

All the embodiments provided above for the aptamer, complex and composition of the invention are also embodiments of the fifth aspect of the invention.

In a particular embodiment of the fifth aspect, the aptamer comprises or consists of sequence SEQ ID NO: 1, SEQ ID NO: 2, SEQ ID NO: 3 or SEQ ID NO: 4, and the complex comprises or consists of sequence SEQ ID NO: 17.

In a sixth aspect, the present invention refers to the aptamer, the complex or the composition according to any one of the embodiments of the invention, for use in a method of diagnosis in vivo of cancer or cancer metastasis, wherein the cancer is an EphA2 expressing cancer.

The sixth aspect also includes an in vivo method of diagnosis of a cancer or cancer metastasis characterised by expressing EphA2 in a subject, the method comprising administering a RNA aptamer, complex according to any one of the embodiments of the second aspect of the invention, or composition as defined in the invention to a subject in need thereof.

All the embodiments provided above for the aptamer, complex and composition of the invention are also embodiments of the sixth aspect of the invention.

In a particular embodiment of the sixth aspect, the aptamer comprises or consists of sequence SEQ ID NO: 1, SEQ ID NO: 2, SEQ ID NO: 3 or SEQ ID NO: 4, and the complex comprises or consists of sequence SEQ ID NO: 17.

In a particular embodiment according to any one of the embodiments of the fourth, fifth and sixth aspects of the invention, the cancer characterised by expressing EphA2 (EphA2 expressing cancer) is a cancer comprising EphA2 positive cell(s). Thus, in a particular embodiment the cancer is a cancer comprising EphA2 positive cell(s). Likewise, in another particular embodiment, the cancer is a cancer overexpressing EphA2. Overexpressing EphA2 means that the expression of EphA2 is at least 2, 3, 4 or 5 times higher than the EphA2 expression in healthy tissues.

Preferably, the EphA2 expressing cancer is selected from the group consisting of soft tissue and bone sarcomas, in particular TAS, such as ES, ARMS, SS, Ewing-like sarcomas (CIC-, BCOR- and EWSR1- rearranged with non-ETS genes), DSRCT, MLS; embryonal rabdomiosarcoma; osteosarcoma; breast cancer, in particular triple negative breast cancer; colorectal cancer; melanoma; renal cell carcinoma; pancreatic cancer; prostate cancer, and combinations thereof. More preferably, the EphA2 expressing cancer is selected from the group consisting of soft tissue and bone sarcoma, in particular TAS, such as ES, ARMS, SS; Ewing-like sarcomas (CIC—, BCOR—and EWSR1- rearranged with non-ETS genes); DSRCT, MLS; osteosarcoma; breast cancer, in particular triple negative breast cancer; colorectal cancer; melanoma; renal cell carcinoma; pancreatic cancer; prostate cancer, and combinations thereof. More particularly, the EphA2 expressing cancer is a TAS, preferably ES, ARMS, SS; Ewing-like sarcomas (e.g., CIC—, BCOR—and EWSR1- rearranged with non-ETS genes), DSRCT, MLS, or breast cancer, preferably triple negative breast cancer. Even more preferably the cancer is ES, ARMS or SS.

The dosage of administration of the aptamer, complex, or composition of the present invention varies depending on the kind and activity of active ingredient, seriousness of disease, subject of administration, drug tolerability of the subject of administration, body weight, age and the like, and the usual dosage, based on the amount of active ingredient per day for an adult, can be about 0.0001 to about 100 mg/kg, for example, about 0.0001 to about 10 mg/kg, preferably about 0.005 to about 1 mg/kg.

The aptamer, complex and/or composition of the present invention can be comprised within a kit of parts. Thus, a seventh aspect of the invention refers to a diagnostic kit comprising the aptamer according to any one of the embodiments of the first aspect of the invention, the complex according to any one of the embodiments of the second aspect of the invention, and/or the composition according to any one of the embodiments of the third aspect of the invention. It also refers to the use of this kit for in vitro or ex vivo diagnosis of cancer or cancer metastasis, wherein the cancer is characterised by expressing EphA2. Preferably the kit comprises means to detect the aptamer. More preferably, the kit comprises instructions for its use.

All the embodiments provided above for the aptamer, complex or composition are also embodiments of the seventh aspect of the invention.

In one embodiment of the seventh aspect of the invention, the aptamer comprises or consists of sequence SEQ ID NO: 1, SEQ ID NO: 2, SEQ ID NO: 3 or SEQ ID NO: 4 and the complex comprises or consists of sequence SEQ ID NO: 17.

While the foregoing invention has been described in some detail for purposes of clarity and understanding, it will be appreciated by one skilled in the art from a reading of this invention that various changes in form and detail can be made without departing from the true scope of the invention and appended claims.

The examples below serve to further illustrate the invention and are not intended to limit the scope of the present invention.

### EXAMPLES

### Example 1.—Material and Methods

### 1.—Cell-Internalization SELEX

An RNA library with a 30-nucleotide (nt) variable region (gggaggacgaugcggnnnnnnnnnnnnnnnnnnnnnnnnnnnnnncagacgacucgcccga, SEQ ID NO 23) was generated by in vitro transcription using a mutant Y639F T7 RNA polymerase and chemically synthesized DNA templates (IDT). The in vitro transcription reactions for the library and all subsequent rounds of SELEX were supplemented with 2′-fluoro modified CTP and UTP (TriLink Biotechnologies) to generate RNAs that are nuclease-resistant.

In each round of cell SELEX, RNA aptamer pools (150 nM) supplemented with 100 g/ml yeast tRNA (Invitrogen) were first incubated on non-target MCF10A (EphA2-) cells for 30 min to remove aptamers that bind to and are internalized into the non-target cells. Next, the supernatant (containing RNA aptamers that do not internalize into the non-target cells) was transferred to target MDA-MB 231 (EphA2+) cells for 30 min. To increase the stringency of the selection in later rounds of cell-internalization SELEX, internalization time and number of cells were reduced. To remove unbound and surface-bound aptamers, target cells (MDA-MB 231) were washed with ice-cold DPBS adjusted to 0.5 M NaCl (High Salt Wash) for 5 min. Internalized RNA aptamers were then recovered using TRIzol reagent (Invitrogen) following manufacturer's instructions, reverse transcribed into DNA, amplified by PCR (Sel2 5′ primer: taatacgactcactatagggaggacgatgcgg, SEQ ID NO 24; Sel2 3′ primer: tcgggcgagtcgtctg, SEQ ID NO 25), and in vitro transcribed to generate an enriched pool of RNA aptamers for the next round of cell-internalization SELEX.

Pools of aptamers from select human EphA2 rounds were sequenced using 70 Illumina deep sequencing (Iowa State DNA Facility). To determine the percent enrichment, the total number of unique sequences in each round was divided by the total number of sequences obtained in each round. Aptamers were grouped into families by comparing each individual aptamer sequence with all others in the selection. The most highly represented aptamer was used to test its ability to enter the cells.

The sequence of the aptamer used in all the Examples is: gggaggaCgaUgCggUCCUUgUCgUCUUgCgUCCCCagaCgaCUCgCCCga (SEQ ID NO: 4), wherein capital letter denotes 2′-fluoro modified base.

### 2.—Internalization Assay

Target (A673 and SKNMC) cells were incubated with 100 nM aptamer or aptamer-siRNA chimera for 30 min at 37 °C with 5% CO₂. Cells were washed with ice-cold High Salt Wash and RNA was recovered using TRIzol reagent. Samples were normalized to an internal RNA reference control. Specifically, 0.5 pmol/sample M12-23 aptamer was added to each sample along with TRIzol as a reference control. Recovered RNAs were quantitated using iScript One-Step RT-PCR Kit with SYBR Green (Biorad) with a Biorad iCycler. All reactions were done in a 50 μl volume in triplicate with primers specific for RNA aptamers (Sel2 5′ primer (SEQ ID NO 24); Sel 2 3′ primer (SEQ ID NO 25) and M12-23 reference control (Sel1 5′ primer: gggggaattctaatacgactcactatagggagagaggaagagggatggg, SEQ ID NO 26; Sel 1 3′ primer: ggggggatccagtactatcgacctctgggttatg, SEQ ID NO 27)). Samples were normalized to M12-23, as well as the PCR amplification efficiency of each aptamer relative to SCR1 control aptamer.

### 3.—RNA Extraction and Reverse Transcription

After aptamer or chimera treatment at the stated concentration, total RNA (2 g), extracted by using the nucleoSpin RNA or the NucleoSpin miRNA (for Microarray purpose) from Macherey-Nagel, was used for cDNA synthesis with SuperScript II Reverse Transcriptase (Life Technologies).

4.—Quantitative Real Time PCR (qPCR)

Quantitative reverse transcription-PCR was performed under universal cycling conditions on LightCycler 480 II instrument (Roche) using TaqMan PCR Mastermix and TaqMan probes from Life Technologies.

### Example 2.—Expression of EPHA2

Cells were lysed with RIPA Buffer (Thermo Fisher Scientific, Waltham, Massachusetts, USA) containing protease inhibitors (Complete, Mini; Protease Inhibitor Cocktail Tablets, Roche) and phosphatase inhibitors (PhosStop, Phosphatase Inhibitor Cocktail Tablets, Roche) for 30 min on ice. Lysates were sonicated, centrifuged at 13,000 rpm at 4 °C for 30 min, and supernatants recovered. Samples (50 μg) were resolved by 8, 10 or 12% SDS-PAGE and transferred onto nitrocellulose membranes (0.2 μm, Bio-Rad, Hercules, California, USA). Membrane blocking was performed with 5% skimmed milk in PBS containing 0.1% Tween20 (Sigma-Aldrich) at room temperature for 1 hr. Next, membranes were incubated overnight at 4 °C with the appropriate primary antibody (EphA2 1:1,000 #6997). Blots were then incubated at room temperature for 1 hr with a horseradish peroxidase-conjugated secondary antibody (goat anti-rabbit, Life Technologies) and the peroxidase activity was detected by enhanced chemiluminescence (Thermo Fisher Scientific) following the manufacturer's instructions. Immunodetection of α-tubulin (#ab28439) or β-actin (#ab49900) from Abcam was used as a loading control.

As shown in FIGS. 1A, 1B, and 1C, EphA2 is highly expressed in RMS cells (FIG. 1A). Moreover, stable knockdown of EphA2 in RH4 cells (FIG. 1B) results in reduction of the neoplastic phenotype of these cells especially on migration (FIG. 1C). Thus, EphA2 is overexpressed in RMS cells and its downregulation results in reduction of cell migration.

### Example 3.—Recognition and Internalization

A673 cells that express EphA2 were treated with 100 nM scramble aptamer, an unspecific RNA sequence or the EphA2 specific aptamer. Cells were fixed and an immunofluorescence for EphA2 was performed. Green stains EphA2 on the membranes of cells, DAPI stains nuclei and the red color results from the Cy3 tag attached to the EphA2 aptamer that has internalized the cells. Pictures were taken 3 hours after treatment.

It was found that the EphA2 aptamer of the present invention recognized and entered ES A673 cells (EphA2-expressing cells) as cells were red stained. Thus, it is demonstrated the ability of the aptamer of the invention to recognize and internalize EphA2 positive cells. Like this, the aptamer of the present invention is a perfect therapeutic candidate and delivery agent of any functional substance coupled to it to said EphA2 positive cells.

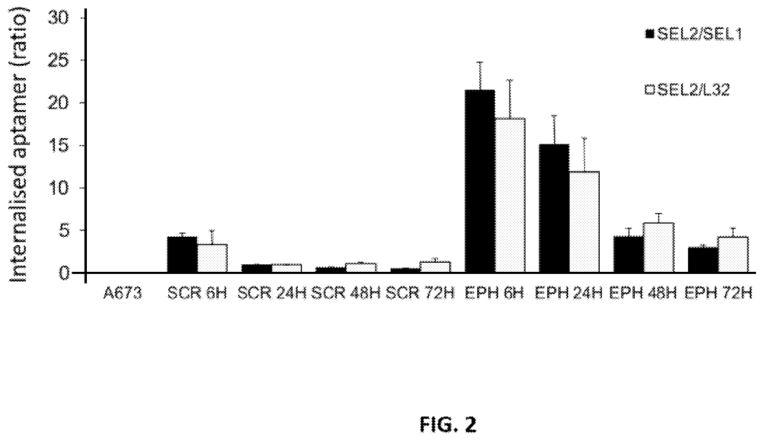

Moreover, EphA2 (EPH) and Scrambled (SCR) RNA aptamers were incubated with ES EphA2+A673 cells. The RNAs that internalized into the cells were recovered by TRIzol extraction and quantified using qPCR after the indicated time points (FIG. 2). SCR RNA aptamer was used as negative controls for cell-internalization in this assay. As predicted, the EphA2 RNA aptamer internalized specifically into A673 cells with a peak at 6 h and little-to-no internalization was observed using the SCR RNA aptamer. Black bars represent internalized RNA specific from the aptamer (as specific primers of the generating library, SEL2/SEL1 were used), light grey bars relate to the ratio of the specific primer and internal RNA from the cell (L32 represents RNA from a ribosomal protein present in the cell).

### Example 4.—Clonogenic Assay

After demonstrating the ability of the aptamer to internalize the cells, the inventors tested whether it may have any therapeutic effect by clonogenic assays.

For clonogenic assays, 500 cells were seeded in the wells of a 6-well plate. When colonies reached saturation, approximately 14 days after seeding, cells were fixed with cold methanol for 10 min, washed with Dulbecco's Phosphate Buffered Saline (PBS, Biowest), stained with crystal violet (Sigma-Aldrich) for 20 min, and washed with water. The total colony number was manually counted using ImageJ. In some cases, colonies were discolored with a 10% glacial acetic acid solution and crystal violet was quantified by spectrometry.

ES cells: A673 (A6) and TC252 (TC2), and ARMS cells: RH4 and RMS13, were treated with either scramble (SCR) or EphA2 aptamer (EPH) at 100 nM every 3 days for 14 days. FIGS. 3A and B show a representative experiment of the number of stained colonies in SCR and EphA2 aptamer treated cells, respectively. Graphic of FIG. 3C shows number of colonies as a median percentage counted in each cell line (×3). In comparison to the scramble aptamer, the EphA2 aptamer of the present invention was able to reduce the clonogenic capacity of cells representative of ES and ARMS entities (FIG. 3C).

### Example 5.—Transwell Migration Assay

Migration assay was performed on A673 (ES) and RMS13 (ARMS) cells treated with either scramble or EphA2 aptamer.

Cells were harvested as usual. After an additional wash with RPMI, 1.5×10⁵ cells in 150 μL serum-free medium were added to the top chamber of 8-μm pore polycarbonate transwells (Transwell Permeable Supports-Corning). Meanwhile, in the bottom chamber, 500 μL of complete medium (10% FBS) were added. For the migration assays in the presence of 250 nM aptamer, cells were pre-treated with the aptamer the 6 h prior seeding and the aptamer was added to both chambers. After 48 h for A673 and 6 h for RMS13, cells on the upper chamber were removed with a cotton swab. Migrating cells still attached on the membrane's underside were fixed for 30 min using 70% ethanol and stained with crystal violet.

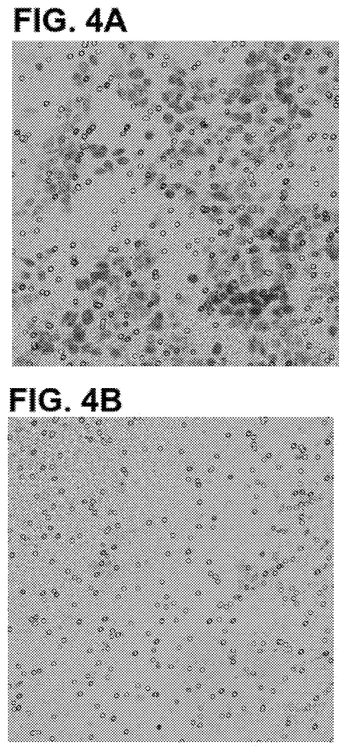

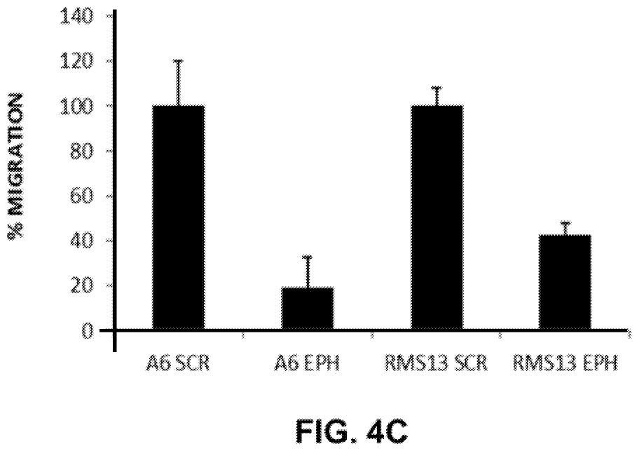

Representative micrographs of migrated A673 cells after scramble and EphA2 aptamer treatment are shown in FIGS. 4A and 4B, respectively.

Transwell membranes were collected and 5 pictures of each transwell were acquired by optical microscopy (100×). Generally, membranes were discolored with a 10% glacial acetic acid solution and crystal violet was quantified by spectrometry. In some instances, we opted for a direct manual counting of the number of migrating cells in the membrane using ImageJ. Results are presented as the percentage of a designated control condition (FIG. 4C).

As shown in FIG. 4C, the aptamer of the present invention reduces migration of both ES and RMS cells, strongly suggesting that the aptamer mimics the effects of knocking down EphA2.

### Example 6.—Tumor Incidence

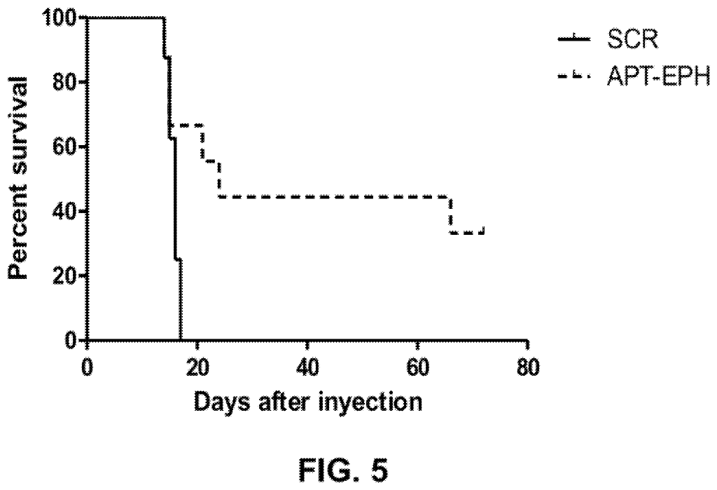

Based on the in vitro results, the inventors tested the effects of the aptamer in vivo by using an orthotopic model developed by the inventors (Lagares-Tena et al.). A673 cells (2×10⁶) were injected in the gastrocnemius of balb/c female mice (8 mice for scramble treatment and 9 mice for EphA2 aptamer treatment) and 2 days after, scramble and EphA2 aptamer were applied systematically through the tail vein every 3 days at a 2 nmol concentration (4 to 5 injections were applied). As shown in FIG. 5, all scramble treated mice developed tumors right to surgery by 18 days. In contrast, as a consequence of the treatment, tumors did not develop in 3 out of the 9 mice treated with the specific aptamer and the tumors developed in four of the other mice had a significant growth delay. Moreover, the time to reach the volume for surgery was delayed.

### Example 7.—Orthotopic Xenograft Metastasis Assay

As the orthotopic model develops lung metastasis after tumor excision, the inventors measured the number of lung metastases in each group of animals.

Briefly, 2×10⁶ cells resuspended in 100 L of PBS were injected into the gastrocnemius muscles of 6-week-old female athymic nude mice (BALB/cnu nu) from Harlan (8 mice for scramble treatment and 9 mice for EphA2 aptamer treatment) and 2 days after, scramble and EphA2 aptamer were applied systematically through the tail vein every 3 days at a 2 nmol concentration (4 to 5 injections were applied). Once primary tumor-bearing limbs reached a volume of 800 mm³, the gastrocnemius muscles were surgically resected. At day 60 after injection, mice were euthanized, and lungs were fixed in 4% paraformaldehyde and embedded in paraffin. Lung sections were stained with hematoxylin & eosin and metastases were counted under an optical microscope.

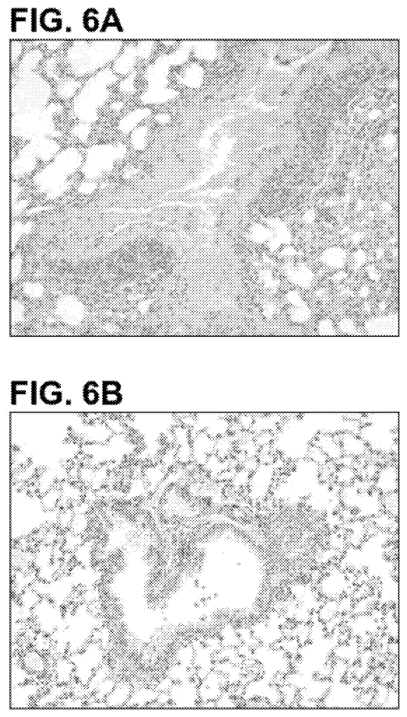

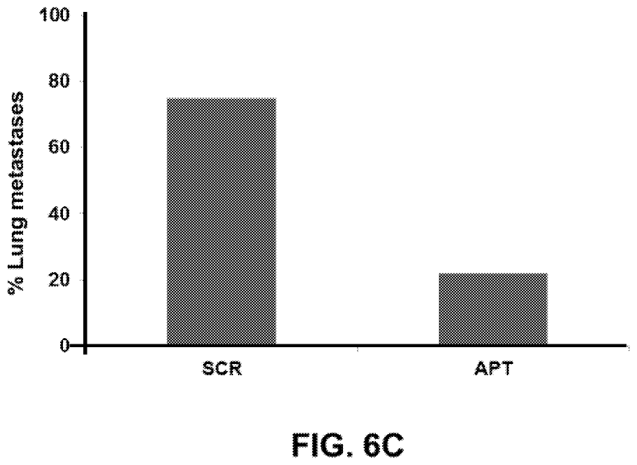

Micrographs representative of a lung micrometastasis in scramble-treated mice and healthy lung from EphA2 aptamer-treated mice are shown in FIGS. 6A and 6B, respectively.

As seen in FIG. 6C, only 2 mice treated with the EphA2 aptamer showed micrometastases in the lungs, representing 28% of the sample. In contrast, in 7 mice treated with the scramble aptamer micrometastases were found in the lungs, representing 77% of the sample.

### Example 8.—Aptamer-siRNA Complex

### Chimera Generation

The longer strands of the EphA2 aptamer-EWS/FLI1 siRNA chimeras were engineered by adding nucleotides complementary to the EWS/FLI1 antisense sequence to the 3′ termini of the EphA2 RNA aptamers (underlined in SEQ ID NO 17, below). A linker uuu was included between the aptamer and the siRNA and a tail uu was included at the 3′end of the siRNA (italic in SEQ ID NO 17, below). All RNAs generated by in vitro transcription were produced with 2′-fluoro modified pyrimidines (capital letters in the sequence) to render the RNAs resistant to nuclease degradation. A 4-fold molar excess of the EWS/FLI1 antisense sequence was annealed to each long RNA strand (at a final concentration of 1 μM) by heating the long RNA strand at 95 °C for 10 min, adding the 4-fold excess antisense siRNA strand to the unfolded aptamer solution and transferring the mixture to a 65 °C dry bath for 7 min. The RNA mixture was allowed to cool at 25 °C for 20 min to allow annealing of the two RNA strands. RNA aptamers and siRNAs were then folded and annealed in 1×BB (20 mM HEPES pH 7.4, 150 mM NaCl, 2 mM CaCl₂)). The excess antisense siRNA strand was removed by filtering the folded RNAs through Amicon Y-30 columns (Millipore, UFC803024).

The chimera used in this Example was:

(SEQ ID NO: 17)
gggaggaCgaUgCggUCCUUgUCgUCUUgCgUCCCCagaCgaCUCgCCCg
auuuCgggCagCagaaCCCUUCUUaUgaCuu

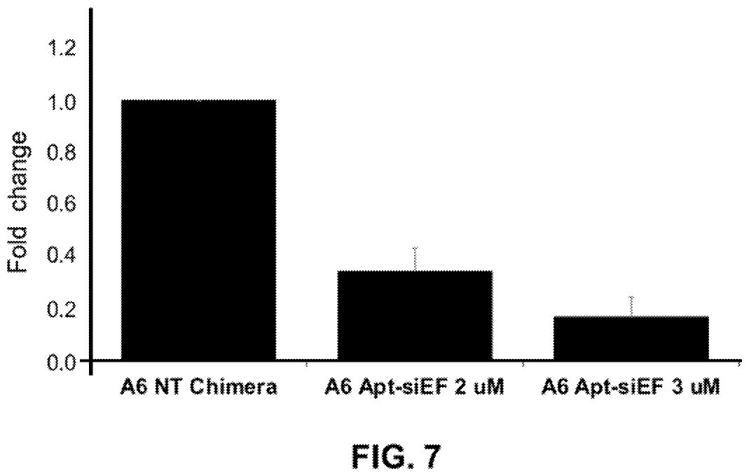

A673 cells were treated for 48 h with a non-targeting (NT) chimera and the specific chimera (Apt-siEF) at different concentrations without using any lepidic system. EWS/FLI1 expression was measured by qPCR using TaqMan probes from Life Technologies ACTB 4333762F and EWS-FLI1 Hs03024497. Levels of the fusion gene lowered around 80% after siRNA delivery (see FIG. 7). Thus, A673 cells treated for 48 h with this EPhA2-specific aptamer complexed with siRNA for EWS/FLI1 results in an efficient downregulation of EWS/FLI1.

This Example shows that an aptamer-siRNA complex according to the present invention is able to internalize into specific cell types (EphA2 positive cells) and can deliver functional substances (in this case siRNA specific for EWS/FLI1) into cells in vitro, resulting in the down-regulation of the expression of the target gene of the siRNA (EWS/FLI1 in this case). Thus, it is proved that the aptamer of the present invention is a good delivery agent, which allows the internalization of the siRNA complexed to it, and protects said siRNA from its degradation.

These results strongly suggest the usefulness of the aptamer according to the invention delivering specific siRNAs into cells that efficiently are processed to inhibit the expression of the siRNA target.

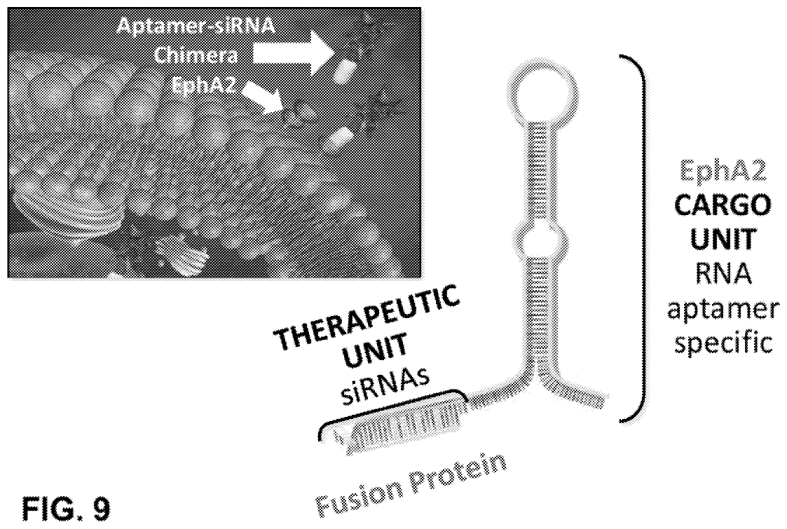

A hypothetic model about how the EphA2 aptamer-siRNA chimera works at the cellular level is depicted in FIG. 9. The aptamer-siRNA chimera recognizes the receptor in the plasmatic membrane and enters the cell.

### Example 9.—Clonogenic Assay Using a Complex of the Invention

The same protocol as the one disclosed in Example 4 above was followed but replacing the aptamer by the complex of sequence SEQ ID NO: 17 of Example 8 and testing the effect on A673 cells.

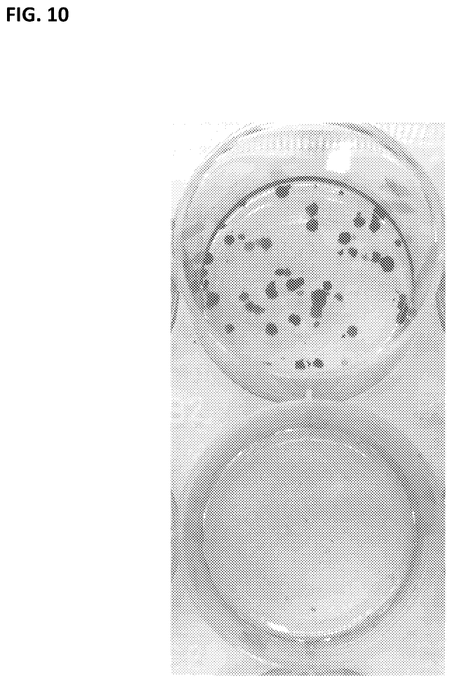

The results are summarized in FIG. 10. It is clear that the complex is remarkably efficient in reducing the clonogenic capacity of ES cells.

### LITERATURE

Xiao et al., Advances in chromosomal translocations and fusion genes in sarcomas and potential therapeutic applications. Cancer Treat Rev. 2018; 63: 61-70.
Tandon et al., Emerging strategies for EphA2 receptor targeting for cancer therapeutics. Expert Opin Ther Targets. 2011; 15(1): 31-51.
Kasinski and Slack, Small RNAs deliver a blow to ovarian cancer. Cancer Discov. 2013; 3: 1220-1221.
Quinn et al., Therapy of pancreatic cancer via an EphA2 receptor-targeted delivery of Gemcitabine. Oncotarget. 2016; 7: 17103-17110.
Garcia-Monclns et al., EphA2 receptor is a key player in the metastatic onset of Ewing sarcoma. Int. J. Cancer. 2018; 143: 1188-1201.
Lagares-Tena et al. Caveolin-1 promotes Ewing sarcoma metistasis regulating MMP-9 expression through MAPK/ERK pathway. Oncotarget. 2016; 7: 56889-56903.
Dassie et al. Systemic administration of optimized aptamer-siRNA chimeras promotes regression of PSMA-expressing tumors. Nat Biotechnol. 2009; 27(9): 839-49.
Cheng and Saltzman. Enhanced siRNA delivery into cells by exploiting the synergy between targeting ligands and cell-penetrating peptides. Biomaterials 2011; 32(26):6194-203.
Zhou Y. et al., “Emerging and Diverse Functions of the EphA2 Noncanonical Pathway in Cancer Progression”, Biol. Pharm. Bull. 40, 1616-1624 (2017).

For reasons of completeness, various aspects of the invention are set out in the following numbered clauses:

### CLAUSES

1.—An RNA-aptamer which binds specifically to EphA2 and which:
(i) consists of sequence SEQ ID NO: 1; or
(ii) comprises sequence SEQ ID NO 2, optionally comprising one, two or three substitutions located within any of the positions 1-20 and 46-51 of SEQ ID NO 2.
2.—The aptamer according to clause 1, wherein the aptamer is modified to protect it from nuclease digestion, preferably modified by comprising pyrimidine bases 2′-fluoro (2′-F) modified or by coupling polyethyleneglycol to the 5′-end of the aptamer.
3.—The aptamer according to clause 2, wherein the aptamer comprises the pyrimidine bases 2′-fluoro (2′-F) modified and
(i) consists of sequence SEQ ID NO: 3; or
(ii) comprises sequence SEQ ID NO: 4, optionally comprising one, two or three substitutions located within any of the positions 1-20 and 46-51 of SEQ ID NO: 4.
4.—The aptamer according to any one of clauses 1 to 3, comprising or consisting of sequence SEQ ID NO: 2 or SEQ ID NO: 4, preferably SEQ ID NO: 4.
5.—A complex comprising the RNA-aptamer according to any one of clauses 1 to 4, coupled to a functional substance, preferably coupled at the 3′end of the aptamer.
6.—The complex according to clause 5, wherein the functional substance is coupled to the aptamer by a spacer, preferably a spacer of 2-5 nucleotides, more preferably of 3 nucleotides, and/or wherein the functional substance comprises a 3′-end tail, preferably a tail of 2-5 nucleotides, more preferably of 2 or 3 nucleotides.
7.—The complex according to clause 5 or 6, wherein the functional substance is:
(i) an siRNA, microRNA, shRNA or a ribozyme, preferably siRNA or microRNA; or
(ii) a moiety selected from a radionuclide, a chemotherapeutic agent and combinations thereof, preferably a chemotherapeutic agent.
8.—The complex according to clause 7, wherein the aptamer is coupled to an siRNA, and preferably said siRNA comprises any one of sequences SEQ ID NO: 5 to SEQ ID NO 10.
9.—The complex according to clause 7, comprising any one of sequences SEQ ID NO 11 to SEQ ID NO 22.
10.—The complex according to clause 5 or 6, wherein the functional substance is a detectable label, preferably selected from the group consisting of an enzyme, prosthetic group, fluorescent material, luminescent material, bioluminescent material, electron dense label, labels for magnetic resonance imaging, radioactive material, and combinations of these.
11.—A composition comprising the aptamer according to any one of clauses 1 to 4, and/or the complex according to any one of clauses 5 to 10, and a pharmaceutically and/or physiological acceptable carrier.
12.—The aptamer according to any one of clauses 1 to 4, or the complex according to any one of clauses 5-10, or the composition according to clause 11, for use in a method of treating or preventing cancer or cancer metastasis in a subject, wherein the cancer is characterised by expressing EphA2, preferably the cancer is selected from the group consisting of soft tissue and bone sarcoma, in particular translocation-associated sarcoma, such as Ewing sarcoma, alveolar rhabdomyosarcoma, synovial sarcoma; Ewing-like sarcomas; osteosarcoma; breast cancer, such as triple negative breast cancer; colorectal cancer; melanoma; renal cell carcinoma; pancreatic cancer; prostate cancer, and combinations thereof.
13.—Use of the aptamer of any one of clauses 1 to 4, or the complex according to any one of clauses 10, or the composition according to clause 11 for in vitro or ex vivo diagnosis of cancer or cancer metastasis, wherein the cancer is characterised by expressing EphA2, preferably the cancer is selected from the group consisting of soft tissue and bone sarcoma, in particular translocation-associated sarcoma, such as Ewing sarcoma, alveolar rhabdomyosarcoma, synovial sarcoma; Ewing-like sarcomas; osteosarcoma; breast cancer, in particular triple negative breast cancer; colorectal cancer; melanoma; renal cell carcinoma; pancreatic cancer; prostate cancer, and combinations thereof.
14.—The RNA aptamer according to any one of clauses 1 to 4, or the complex according to any one of clauses 10, or the composition according to clause 11, for use in a method of diagnosis in vivo of a cancer characterised by expressing EphA2, preferably the cancer is selected from the group consisting of soft tissue and bone sarcoma, in particular translocation-associated sarcoma, such as Ewing sarcoma, alveolar rhabdomyosarcoma, synovial sarcoma; Ewing-like sarcomas; osteosarcoma; breast cancer, in particular triple negative breast cancer; colorectal cancer; melanoma; renal cell carcinoma; pancreatic cancer; prostate cancer, and combinations thereof.
15.—Diagnostic kit comprising the RNA aptamer according to any one of clauses 1 to 4, or the complex according to any one of clauses 10, or the composition according to clause 11, and optionally comprising means to detect the aptamer.

1. A method of treating or preventing cancer or cancer metastasis in a subject, wherein the cancer is characterized by expressing EphA2, the method comprising administering to a subject in need thereof, a therapeutically effective amount of:
an RNA-aptamer which binds specifically to EphA2 and consists of sequence SEQ ID NO: 4;
a complex comprising the aptamer coupled to an siRNA; or
a pharmaceutical composition comprising the RNA-aptamer or the complex at a therapeutically effective amount together with a pharmaceutically acceptable excipient and/or carrier.

2. The method according to claim 1, wherein the cancer is Ewing sarcoma, Ewing-like sarcoma, or alveolar rhabdomyosarcoma.

3. The method according to claim 1, wherein the RNA-aptamer is coupled to the siRNA through a spacer.

4. The method according to claim 3, wherein part or all of the nucleotides forming the spacer are uracil nucleotides.

5. The method according to claim 1, wherein at least one of the nucleotides forming part of the siRNA is a modified nucleotide.

6. The method according to claim 1, wherein the siRNA comprises the sequence SEQ ID NO: 5.

7. The method according to claim 1, wherein the siRNA comprises a 3′-end nucleotide tail.

8. The method according to claim 7, wherein the nucleotide tail is formed by 2 to 5 nucleotides.

9. The method according to claim 7, wherein the nucleotide tail is formed by 2 or 3 nucleotides.

10. The method according to claim 7, wherein the nucleotide tail comprises or consists of uracil nucleotides.

11. The method according to claim 1, wherein the complex comprises or consists of the sequence SEQ ID NO: 17.


SEQUENCE LISTING (27 sequences)

SEQ ID NO: 1 - RNA, 25 nt, synthetic construct
  misc_feature 1..25: note = Aptamer
  source 1..25: mol_type = other RNA
gtcgtcttgcgtccccagacgactc

SEQ ID NO: 2 - RNA, 51 nt, synthetic construct
  misc_feature 1..51: note = Aptamer
  source 1..51: mol_type = other RNA
gggaggacgatgcggtccttgtcgtcttgcgtccccagacgactcgcccga

SEQ ID NO: 3 - RNA, 25 nt, synthetic construct
  misc_feature 1..25: note = Modified aptamer
  modified_base 2..3: mod_base = OTHER; note = 2'-fluoro nucleotide
  modified_base 5..8: mod_base = OTHER; note = 2'-fluoro nucleotide
  modified_base 10: mod_base = OTHER; note = 2'-fluoro nucleotide
  modified_base 12..16: mod_base = OTHER; note = 2'-fluoro nucleotide
  modified_base 20: mod_base = OTHER; note = 2'-fluoro nucleotide
  modified_base 23..25: mod_base = OTHER; note = 2'-fluoro nucleotide
  source 1..25: mol_type = other RNA
gtcgtcttgcgtccccagacgactc

SEQ ID NO: 4 - RNA, 51 nt, synthetic construct; identical to SEQ ID NO: 2
  misc_feature 1..51: note = Modified aptamer
  modified_base 8: mod_base = OTHER; note = 2'-fluoro nucleotide
  modified_base 11: mod_base = OTHER; note = 2'-fluoro nucleotide
  modified_base 13: mod_base = OTHER; note = 2'-fluoro nucleotide
  modified_base 16..20: mod_base = OTHER; note = 2'-fluoro nucleotide
  modified_base 22..23: mod_base = OTHER; note = 2'-fluoro nucleotide
  modified_base 25..28: mod_base = OTHER; note = 2'-fluoro nucleotide
  modified_base 30: mod_base = OTHER; note = 2'-fluoro nucleotide
  modified_base 32..36: mod_base = OTHER; note = 2'-fluoro nucleotide
  modified_base 40: mod_base = OTHER; note = 2'-fluoro nucleotide
  modified_base 43..45: mod_base = OTHER; note = 2'-fluoro nucleotide
  modified_base 47..49: mod_base = OTHER; note = 2'-fluoro nucleotide
  source 1..51: mol_type = other RNA

SEQ ID NO: 5 - RNA, 25 nt, synthetic construct
  misc_feature 1..25: note = siRNA
  source 1..25: mol_type = other RNA
cgggcagcagaacccttcttatgac

SEQ ID NO: 6 - RNA, 25 nt, synthetic construct
  misc_feature 1..25: note = siRNA
  source 1..25: mol_type = other RNA
atggcctctcacctcagaattcaat

SEQ ID NO: 7 - RNA, 25 nt, synthetic construct
  misc_feature 1..25: note = siRNA
  source 1..25: mol_type = other RNA
tgcccaagaagccagcagaggaatt

SEQ ID NO: 8 - RNA, 25 nt, synthetic construct
  misc_feature 1..25: note = Modified siRNA
  modified_base 1: mod_base = OTHER; note = 2'-fluoro nucleotide
  modified_base 5: mod_base = OTHER; note = 2'-fluoro nucleotide
  modified_base 8: mod_base = OTHER; note = 2'-fluoro nucleotide
  modified_base 13..20: mod_base = OTHER; note = 2'-fluoro nucleotide
  modified_base 22: mod_base = OTHER; note = 2'-fluoro nucleotide
  modified_base 25: mod_base = OTHER; note = 2'-fluoro nucleotide
  source 1..25: mol_type = other RNA
cgggcagcagaacccttcttatgac

SEQ ID NO: 9 - RNA, 25 nt, synthetic construct
  misc_feature 1..25: note = Modified siRNA
  modified_base 2: mod_base = OTHER; note = 2'-fluoro nucleotide
  modified_base 5..10: mod_base = OTHER; note = 2'-fluoro nucleotide
  modified_base 12..15: mod_base = OTHER; note = 2'-fluoro nucleotide
  modified_base 20..22: mod_base = OTHER; note = 2'-fluoro nucleotide
  modified_base 25: mod_base = OTHER; note = 2'-fluoro nucleotide
  source 1..25: mol_type = other RNA
atggcctctcacctcagaattcaat

SEQ ID NO: 10 - RNA, 25 nt, synthetic construct
  misc_feature 1..25: note = Modified siRNA
  modified_base 1: mod_base = OTHER; note = 2'-fluoro nucleotide
  modified_base 3..5: mod_base = OTHER; note = 2'-fluoro nucleotide
  modified_base 12..13: mod_base = OTHER; note = 2'-fluoro nucleotide
  modified_base 16: mod_base = OTHER; note = 2'-fluoro nucleotide
  modified_base 24..25: mod_base = OTHER; note = 2'-fluoro nucleotide
  source 1..25: mol_type = other RNA
tgcccaagaagccagcagaggaatt

SEQ ID NO: 11 - RNA, 81 nt, synthetic construct
  misc_feature 1..81: note = Complex aptamer-siRNA
  source 1..81: mol_type = other RNA
gggaggacgatgcggtccttgtcgtcttgcgtccccagacgactcgcccgatttcgggcagcagaacccttcttatgactt

SEQ ID NO: 12 - RNA, 55 nt, synthetic construct
  misc_feature 1..55: note = Complex aptamer-siRNA
  source 1..55: mol_type = other RNA
gtcgtcttgcgtccccagacgactctttcgggcagcagaacccttcttatgactt

SEQ ID NO: 13 - RNA, 81 nt, synthetic construct
  misc_feature 1..81: note = Complex aptamer-siRNA
  source 1..81: mol_type = other RNA
gggaggacgatgcggtccttgtcgtcttgcgtccccagacgactcgcccgatttatggcctctcacctcagaattcaattt

SEQ ID NO: 14 - RNA, 55 nt, synthetic construct
  misc_feature 1..55: note = Complex aptamer-siRNA
  source 1..55: mol_type = other RNA
gtcgtcttgcgtccccagacgactctttatggcctctcacctcagaattcaattt

SEQ ID NO: 15 - RNA, 81 nt, synthetic construct
  misc_feature 1..81: note = Complex aptamer-siRNA
  source 1..81: mol_type = other RNA
gggaggacgatgcggtccttgtcgtcttgcgtccccagacgactcgcccgattttgcccaagaagccagcagaggaatttt

SEQ ID NO: 16 - RNA, 55 nt, synthetic construct
  misc_feature 1..55: note = Complex aptamer-siRNA
  source 1..55: mol_type = other RNA
gtcgtcttgcgtccccagacgactcttttgcccaagaagccagcagaggaatttt

SEQ ID NO: 17 - RNA, 81 nt, synthetic construct; identical to SEQ ID NO: 11
  misc_feature 1..81: note = Modified complex aptamer-siRNA
  modified_base 8: mod_base = OTHER; note = 2'-fluoro nucleotide
  modified_base 11: mod_base = OTHER; note = 2'-fluoro nucleotide
  modified_base 13: mod_base = OTHER; note = 2'-fluoro nucleotide
  modified_base 16..20: mod_base = OTHER; note = 2'-fluoro nucleotide
  modified_base 22..23: mod_base = OTHER; note = 2'-fluoro nucleotide
  modified_base 25..28: mod_base = OTHER; note = 2'-fluoro nucleotide
  modified_base 30: mod_base = OTHER; note = 2'-fluoro nucleotide
  modified_base 32..36: mod_base = OTHER; note = 2'-fluoro nucleotide
  modified_base 40: mod_base = OTHER; note = 2'-fluoro nucleotide
  modified_base 43..45: mod_base = OTHER; note = 2'-fluoro nucleotide
  modified_base 47..49: mod_base = OTHER; note = 2'-fluoro nucleotide
  modified_base 55: mod_base = OTHER; note = 2'-fluoro nucleotide
  modified_base 59: mod_base = OTHER; note = 2'-fluoro nucleotide
  modified_base 62: mod_base = OTHER; note = 2'-fluoro nucleotide
  modified_base 67..74: mod_base = OTHER; note = 2'-fluoro nucleotide
  modified_base 76: mod_base = OTHER; note = 2'-fluoro nucleotide
  modified_base 79..81: mod_base = OTHER; note = 2'-fluoro nucleotide
  source 1..81: mol_type = other RNA

SEQ ID NO: 18 - RNA, 55 nt, synthetic construct; identical to SEQ ID NO: 12
  misc_feature 1..55: note = Modified complex aptamer-siRNA
  modified_base 2..3: mod_base = OTHER; note = 2'-fluoro nucleotide
  modified_base 5..8: mod_base = OTHER; note = 2'-fluoro nucleotide
  modified_base 10: mod_base = OTHER; note = 2'-fluoro nucleotide
  modified_base 12..16: mod_base = OTHER; note = 2'-fluoro nucleotide
  modified_base 20: mod_base = OTHER; note = 2'-fluoro nucleotide
  modified_base 23..25: mod_base = OTHER; note = 2'-fluoro nucleotide
  modified_base 29: mod_base = OTHER; note = 2'-fluoro nucleotide
  modified_base 33: mod_base = OTHER; note = 2'-fluoro nucleotide
  modified_base 36: mod_base = OTHER; note = 2'-fluoro nucleotide
  modified_base 41..48: mod_base = OTHER; note = 2'-fluoro nucleotide
  modified_base 50: mod_base = OTHER; note = 2'-fluoro nucleotide
  modified_base 53..55: mod_base = OTHER; note = 2'-fluoro nucleotide
  source 1..55: mol_type = other RNA

SEQ ID NO: 19 - RNA, 81 nt, synthetic construct; identical to SEQ ID NO: 13
  misc_feature 1..81: note = Modified complex aptamer-siRNA
  modified_base 8: mod_base = OTHER; note = 2'-fluoro nucleotide
  modified_base 11: mod_base = OTHER; note = 2'-fluoro nucleotide
  modified_base 13: mod_base = OTHER; note = 2'-fluoro nucleotide
  modified_base 16..20: mod_base = OTHER; note = 2'-fluoro nucleotide
  modified_base 22..23: mod_base = OTHER; note = 2'-fluoro nucleotide
  modified_base 25..28: mod_base = OTHER; note = 2'-fluoro nucleotide
  modified_base 30: mod_base = OTHER; note = 2'-fluoro nucleotide
  modified_base 32..36: mod_base = OTHER; note = 2'-fluoro nucleotide
  modified_base 40: mod_base = OTHER; note = 2'-fluoro nucleotide
  modified_base 43..45: mod_base = OTHER; note = 2'-fluoro nucleotide
  modified_base 47..49: mod_base = OTHER; note = 2'-fluoro nucleotide
  modified_base 56: mod_base = OTHER; note = 2'-fluoro nucleotide
  modified_base 59..64: mod_base = OTHER; note = 2'-fluoro nucleotide
  modified_base 66..69: mod_base = OTHER; note = 2'-fluoro nucleotide
  modified_base 74..76: mod_base = OTHER; note = 2'-fluoro nucleotide
  modified_base 79..81: mod_base = OTHER; note = 2'-fluoro nucleotide
  source 1..81: mol_type = other RNA

SEQ ID NO: 20 - RNA, 55 nt, synthetic construct; identical to SEQ ID NO: 14
  misc_feature 1..55: note = Modified complex aptamer-siRNA
  modified_base 2..3: mod_base = OTHER; note = 2'-fluoro nucleotide
  modified_base 5..8: mod_base = OTHER; note = 2'-fluoro nucleotide
  modified_base 10: mod_base = OTHER; note = 2'-fluoro nucleotide
  modified_base 12..16: mod_base = OTHER; note = 2'-fluoro nucleotide
  modified_base 20: mod_base = OTHER; note = 2'-fluoro nucleotide
  modified_base 23..25: mod_base = OTHER; note = 2'-fluoro nucleotide
  modified_base 30: mod_base = OTHER; note = 2'-fluoro nucleotide
  modified_base 33..38: mod_base = OTHER; note = 2'-fluoro nucleotide
  modified_base 40..43: mod_base = OTHER; note = 2'-fluoro nucleotide
  modified_base 48..50: mod_base = OTHER; note = 2'-fluoro nucleotide
  modified_base 53..55: mod_base = OTHER; note = 2'-fluoro nucleotide
  source 1..55: mol_type = other RNA

SEQ ID NO: 21 - RNA, 81 nt, synthetic construct; identical to SEQ ID NO: 15
  misc_feature 1..81: note = Modified complex aptamer-siRNA
  modified_base 8: mod_base = OTHER; note = 2'-fluoro nucleotide
  modified_base 11: mod_base = OTHER; note = 2'-fluoro nucleotide
  modified_base 13: mod_base = OTHER; note = 2'-fluoro nucleotide
  modified_base 16..20: mod_base = OTHER; note = 2'-fluoro nucleotide
  modified_base 22..23: mod_base = OTHER; note = 2'-fluoro nucleotide
  modified_base 25..28: mod_base = OTHER; note = 2'-fluoro nucleotide
  modified_base 30: mod_base = OTHER; note = 2'-fluoro nucleotide
  modified_base 32..36: mod_base = OTHER; note = 2'-fluoro nucleotide
  modified_base 40: mod_base = OTHER; note = 2'-fluoro nucleotide
  modified_base 43..45: mod_base = OTHER; note = 2'-fluoro nucleotide
  modified_base 47..49: mod_base = OTHER; note = 2'-fluoro nucleotide
  modified_base 55: mod_base = OTHER; note = 2'-fluoro nucleotide
  modified_base 57..59: mod_base = OTHER; note = 2'-fluoro nucleotide
  modified_base 66..67: mod_base = OTHER; note = 2'-fluoro nucleotide
  modified_base 70: mod_base = OTHER; note = 2'-fluoro nucleotide
  modified_base 78..81: mod_base = OTHER; note = 2'-fluoro nucleotide
  source 1..81: mol_type = other RNA

SEQ ID NO: 22 - RNA, 55 nt, synthetic construct; identical to SEQ ID NO: 16
  misc_feature 1..55: note = Modified complex aptamer-siRNA
  modified_base 2..3: mod_base = OTHER; note = 2'-fluoro nucleotide
  modified_base 5..8: mod_base = OTHER; note = 2'-fluoro nucleotide
  modified_base 10: mod_base = OTHER; note = 2'-fluoro nucleotide
  modified_base 12..16: mod_base = OTHER; note = 2'-fluoro nucleotide
  modified_base 20: mod_base = OTHER; note = 2'-fluoro nucleotide
  modified_base 23..25: mod_base = OTHER; note = 2'-fluoro nucleotide
  modified_base 29: mod_base = OTHER; note = 2'-fluoro nucleotide
  modified_base 31..33: mod_base = OTHER; note = 2'-fluoro nucleotide
  modified_base 40..41: mod_base = OTHER; note = 2'-fluoro nucleotide
  modified_base 44: mod_base = OTHER; note = 2'-fluoro nucleotide
  modified_base 52..55: mod_base = OTHER; note = 2'-fluoro nucleotide
  source 1..55: mol_type = other RNA

SEQ ID NO: 23 - RNA, 60 nt, synthetic construct
  misc_feature 1..60: note = RNA library variable region
  source 1..60: mol_type = other RNA
ggaggacgatgcggnnnnnnnnnnnnnnnnnnnnnnnnnnnnnncagacgactcgcccga

SEQ ID NO: 24 - DNA, 32 bp, synthetic construct
  misc_feature 1..32: note = Sel2 5' primer
  source 1..32: mol_type = other DNA
taatacgactcactatagggaggacgatgcgg

SEQ ID NO: 25 - DNA, 16 bp, synthetic construct
  misc_feature 1..16: note = Sel2 3' primer
  source 1..16: mol_type = other DNA
tcgggcgagtcgtctg

SEQ ID NO: 26 - DNA, 49 bp, synthetic construct
  misc_feature 1..49: note = Sel1 5' primer
  source 1..49: mol_type = other DNA
gggggaattctaatacgactcactatagggagagaggaagagggatggg

SEQ ID NO: 27 - DNA, 34 bp, synthetic construct
  misc_feature 1..34: note = Sel 1 3' primer
  source 1..34: mol_type = other DNA
ggggggatccagtactatcgacctctgggttatg